(19) United States
(12) Patent Application Publication
(10) Pub. No.: US 2020/0291431 A1
(43) Pub. Date: Sep. 17, 2020

(54) METHOD FOR THE TREATMENT OF CANCERS BY MEANS OF GENETIC NEUROENGINEERING

(71) Applicant: Convocation Co., Ltd., Central (HK)

(72) Inventors: Takahiro Ochiya, Central (HK); Adrian Sack, Central (HK); Hideaki Shiraishi, Central (HK)

(73) Assignee: Convocation Co., Ltd., Central (HK)

(21) Appl. No.: 16/820,316

(22) Filed: Mar. 16, 2020

Related U.S. Application Data
(60) Provisional application No. 62/819,130, filed on Mar. 15, 2019.

(51) Int. Cl. C12N 15/86; A61K 38/16

(52) U.S. Cl. CPC C12N 15/86; C12N 2799/022; A61K 38/162

(57) ABSTRACT

Methods for the treatment of cancer that include the step of administering a viral vector carrying a nerve deleting, nerve ablating or nerve inhibiting payload, the administration leading to the deletion, ablation or inhibition of tumor-specific sympathetic nerves.

### TECHNICAL FIELD

Aspects of the present disclosure relate to biomedical engineering, in particular neuroengineering for the treatment of tumors by means of the inhibition or ablation of tumor infiltrating sympathetic nerves and the simultaneous stimulation and/or accelerated neurogenesis of tumor infiltrating parasympathetic nerves. More particularly, the present disclosure relates to a novel genetic neuroengineering method for the treatment of cancers employing viral vectors to manipulate tumor-infiltrating local autonomic nerves in a tumor tissue-specific and sympathetic or parasympathetic fiber type-specific manner, thereby treating cancers, particularly breast cancer in human subjects, with significantly increased efficacy and decreased risk of side-effects as compared to current methods.

### BACKGROUND

There are a currently a number of established methods and techniques for the treatment of cancer such as radiotherapy, chemotherapy, immunotherapy, hormone therapy, targeted therapies (typically employing small molecule drugs and monoclonal antibodies) and surgery. Though all such methods aim to maximize the targeting of cancerous tissue whilst minimizing potential side-effects, there are currently few methods capable of exerting profound and precisely targeted anti-tumorigenic effects across a wide range of cancers (in both primary and distant metastatic sites) whilst retaining a minimal side-effect profile.

It is known that autonomic nerves infiltrate normal organs to regulate certain of their functions and a growing body of evidence has suggested a link between autonomic nerves and cancer, although the effect of such nerves (and in particular the differential effect of sympathetic vs. parasympathetic nerves) in various cancer types has remained largely unclear; e.g., epidemiological human and experimental animal studies indicate that chronic stress accelerates cancer growth and progression, potentially by sympathetic neural mechanisms. In addition, recent retrospective clinical studies revealed that β-adrenergic receptor blockers reduce recurrence rates and mortality in patients with breast, melanoma, and prostate cancers, although the reported efficacy is relatively small or insignificant and the attendant side-effects are generally thought to outweigh their benefit.

Existing oncologic methods are limited in their ability to precisely target tumor-specific nerve fibers. For example, pharmacological methods (e.g., β-blockers) also have systemic effects on organs and thus their effects on the functions of tumor-infiltrating local nerves cannot be isolated. In addition, peripheral nerves comprise several types of nerve fibers such that surgical resection of a peripheral nerve leads to disruption of all the nerve fibers contained in the nerve, and thus the neural function of a specific fiber type (e.g., efferent sympathetic nerve fibers, efferent parasympathetic nerve fibers, afferent nerve fibers) cannot be isolated.

Consequently, there is a need to develop a novel oncologic technique incorporating the ability to manipulate tumor-infiltrating local autonomic nerves in a precise tumor-specific sympathetic or parasympathetic fiber type-specific manner in a way that provides an efficient and highly targeted method capable of rapid individualization for the treatment of different cancer types (including breast cancers) with minimal risk of side-effects.

### SUMMARY

In breast cancer we have discovered that stimulation of tumor-infiltrating sympathetic nerves accelerate tumor growth and progression whereas; the stimulation of tumor-infiltrating parasympathetic nerves decelerate it. Genetic deletion of tumor-infiltrating sympathetic nerves using the present invention suppressed tumor growth and downregulated the expression of immune checkpoint molecules (i.e., PD-1, PD-L1, and FOXP3) with greater efficacy than α- or β-noradrenergic receptor blockers. Genetic stimulation of tumor-infiltrating parasympathetic nerves also decreased PD-1 and PD-L1 expression. Consistently, in humans, the increased sympathetic nerve density and decreased parasympathetic nerve density in the tumor microenvironment was associated with a poor clinical outcome and correlated with higher expression of immune checkpoint molecules. We therefore conclude that tumor-infiltrating autonomic nerves regulate the progression of breast cancer in a specific, clinically actionable manner, and that local genetic neuroengineering techniques could be a novel and highly effective approach in such treatment.

According to an embodiment, the present invention provides a method for the treatment of cancer comprising the steps of administering a viral vector, such as an adeno-associated virus, carrying a nerve deleting, nerve ablating or nerve inhibiting payload, preferably via injection, the administration leading to the deletion, ablation or inhibition of tumor-specific (tumor infiltrating and/or peritumoral) sympathetic nerves (such as for example may express sympathetic neuronal markers such as tyrosine hydroxylase (TH)⁺ or the neuron-specific marker neurofilament-L).

According to an embodiment, the present invention provides a method for the treatment of cancer comprising the steps of administering a viral vector, such as an adeno-associated virus, carrying a nerve stimulating payload, resulting in the stimulation of tumor-specific (tumor infiltrating and/or peritumoral) parasympathetic nerves.

According to an embodiment, the present invention provides a method for the treatment of cancer comprising the steps of administering a viral vector, such as an adeno-associated virus, carrying a neurogenesis-promoting (eg. neurotrophic) payload, resulting in the increased growth of tumor-specific (tumor infiltrating and/or peritumoral) parasympathetic nerves.

According to an embodiment, the present invention provides a method for the treatment of cancer carried out in combination with the administration a viral vector, such as an adeno-associated virus, carrying a nerve deleting, nerve ablating or nerve inhibiting payload, preferably via injection, the administration leading to the attenuation or blockade of afferent nerve signals from the tumor site to the brain which might act to adversely modulate the autonomic nervous system (eg. by inducing an autonomic stress response) resulting in the disadvantageous stimulation of the sympathetic nervous system.

According to an alternate embodiment, the present invention provides a method for the treatment of cancer whereby the inhibition of tumor-specific sympathetic nerves and/or the stimulation of tumor-specific parasympathetic nerves is achieved by chemogenetic methods. For instance, the inhibition and/or the stimulation of the nerve are achieved via the installation of a genetically engineered receptor in a tumor-specific nerve (eg. as may be achieved by means of a viral vector carrying chemogenetic elements encoding a genetically engineered receptor) such that the nerve may be modulated by an agonist targeting such receptor.

According to an embodiment, the step of administering the viral vector is carried out by intratumoral injection.

According to an embodiment, the method is carried out in combination with other pharmacological agents which beneficially modulate the autonomic nervous system with respect to specific tumor types, for instance GABA, L-theanine, Hypericum perforatum, and Valeriana officinalis.

According to an embodiment, the method is carried out in combination with the administration of anti-seizure medications which act synergistically with the disclosed genetic neuroengineering method in modulating the autonomic nervous system with respect to specific tumor types, for instance Valproate, Carbamazepine, Ethosuximide, Phenytoin, Benzodiazepine, Lamotrigine, Phenobarbital, Levetiracetam, Gabapentin, Pregabalin, Vigabatrin, Topiramate, and Tiagabine.

According to an embodiment, there is a preferential selection for the co-administration of anti-seizure medications where such medications both beneficially modulate the autonomic nervous system with respect to specific tumor types (for example via GABA-ergic action or sodium or calcium channel inactivation or inhibition) and, furthermore in the case of certain such medications (such as Valproate) where they additionally exert anti-tumorigenic effects in virtue of their action as histone deacetylase inhibitors (HDACi).

According to an embodiment, the method is carried out in combination with the administration of an anti-epileptic diet, preferably a medium-chain triglyceride (MCT) diet, proven effective in patients resistant to anti-epileptic medications.

According to an embodiment, the method is carried out in combination with the use of medical instruments which beneficially modulate the autonomic nervous system with respect to specific tumor types, for example by employing ultra-sound to further locally stimulate parasympathetic nerves.

According to an embodiment, the method is carried out in combination with an administration of conventional chemotherapeutic oncologic agents or acceptable pharmacological agents and radiological treatments.

According to an embodiment, the present invention comprises a diagnostic and monitoring step, comprising a tumoral (and/or peritumoral) biopsy analysis (e.g. by histological immunofluorescence staining or other method), an in vivo scanning method (e.g. photon emission computed tomography of neural cascades or other method), anterograde tracing or other acceptable analytic methods to determine the precise extent of peritumoral and intratumoral nerve innervation, nerve density and the precise ratio of sympathetic and parasympathetic nerves. Furthermore, the analysis methods may carried out in efferent and afferent nerves. Accordingly, the present invention further comprises a method determining the subsequent modality of tumor-specific treatment (as to dosage, period of administration and ratio and manner of sympathetic and parasympathetic nerve targeting in accordance with the disclosed oncologic neuroengineering methods) via sympathetic nerve deletion, ablation or inhibition and in the case of certain tumors (eg. breast cancer) parasympathetic nerve stimulation and/or accelerated neurogenesis. The monitoring step may be repeated throughout the course of treatment to continually inform the application of the neuroengineering method in accordance with tumor regression (or progression) and alteration of peritumoral and intratumoral nerve innervation (described above as to the extent of nerve innervation, nerve density and the precise ratio of sympathetic and parasympathetic nerves) in order to continuously maximize anti-tumorigenic effects and minimize the risk of extra-tumoral side-effects.

### BRIEF DESCRIPTION OF THE DRAWINGS

FIGS. 1a-1c illustrate CD4⁺ and CD8⁺ T cells present and expressing β2-adrenergic receptors in tumor microenvironment of mouse xenograft models of human breast cancer, as described in the Examples.

FIGS. 2a-2n illustrate a genetic deletion of tumor-infiltrating sympathetic nerves suppressing immune checkpoints in tumor microenvironment in human breast cancer xenografts, as described in the Examples.

FIGS. 3a-3p illustrate a deletion of tumor-infiltrating sympathetic nerves suppressing immune checkpoints in tumor microenvironment, as described in the Examples.

FIGS. 4a-4d illustrate quantitative PCR and immunofluorescent analyses of adrenergic receptors in chemically-induced breast cancer, as described in the Examples.

FIGS. 5a-5i illustrate arterial baroreflex limits tumor-suppression during chronic stress by injections of β-adrenergic blocker in chemically-induced breast cancer, as described in the Examples.

FIGS. 6a-6n illustrate genetic stimulation of tumor-infiltrating parasympathetic nerves suppresses immune checkpoints in tumor microenvironment of mouse xenograft models of human breast cancer cells, as described in the Examples.

FIGS. 7a-7w illustrate tumor-infiltrating parasympathetic nerves decelerating growth of chemically-induced breast cancer by suppressing immune checkpoints, as described in the Examples.

FIGS. 8a-8i illustrate human breast cancers having abundant sympathetic and few parasympathetic nerve fibers in patients with recurrence, as described in the Examples.

FIGS. 9a-9s illustrate expression of immune checkpoints positively and negatively correlating with sympathetic and parasympathetic nerve density, respectively, in human breast cancer samples, as described in the Examples.

FIGS. 10a-10f illustrate FOXP3 expression and autonomic nerve fibers in human breast cancer samples, as described in the Examples.

FIGS. 11a-11f illustrate expression PD-1 on CD4⁺ and CD8⁺ tumor-infiltrating lymphocytes in human breast cancer samples, as described in the Examples.

FIGS. 12a-12k illustrate the comparison between patients with TNBC and non-TNBC in autonomic nerve densities and immune-related parameters, as described in the Examples.

### DETAILED DESCRIPTION

In one embodiment, the present invention provides a new retrograde virus vector-based genetic neuroengineering technique to manipulate tumor-infiltrating local autonomic nerves in a tumor-specific and sympathetic or parasympathetic fiber type-specific manner. The effect of breast cancer tumor-infiltrating sympathetic and/or parasympathetic nerves on tumor growth and progression was investigated. The present invention provides a method of tumor treatment by directly manipulating nerves in a tumor tissue-specific and nerve fiber type-specific manner.

The terms “deleting,” “ablating,” “inhibiting,” and the likes may be used hereby for the same outcome especially for the genetic deletion of tumor-infiltrating sympathetic nerves, suppressed tumor growth, and downregulated the expression of immune checkpoint molecules (i.e., PD-1, PD-L1, and FOXP3).

In another embodiment, the present invention provides a method of tumor treatment via the simultaneous inhibition or ablation of tumor-infiltrating sympathetic nerves along with the simultaneous stimulation of tumor-infiltrating parasympathetic nerves. The invention comprises a genetic neuroengineering technique employing viral vectors to manipulate tumor-infiltrating local autonomic nerves in a tumor tissue specific and sympathetic or parasympathetic fiber type-specific manner.

In another embodiment, the present invention provides a technique to manipulate autonomic nerves in a tumor-specific and fiber type-specific manner in mice with human breast cancer xenografts and rats with chemically-induced tumors.

One possible mechanism responsible for tumor suppression by genetic neuroengineering of tumor-infiltrating autonomic nerves is inhibition of the immune checkpoint molecules, PD-1, PD-L1, and FOXP3, which strongly suppress anti-tumor immune responses. In the present invention, it is shown that genetic denervation of tumor-infiltrating sympathetic nerves and neurostimulation of parasympathetic nerves suppressed the expression of immune checkpoint molecules in animal models of breast cancer. It is supported by the observed association of a lower density of tumor-infiltrating sympathetic nerves and a greater density of parasympathetic nerve fibers in human breast cancer specimens with reduced expression of the immune checkpoints molecules PD-1, PD-L1, and FOXP3, and a better clinical outcome. The functional link between tumor-infiltrating sympathetic nerves and immune checkpoints was associated with histological observations that tumor-infiltrating sympathetic nerves were frequently in contact with lymphocytes expressing PD-1 or FOXP3, and also surrounded or infiltrated into tumor tissue expressing PD-L1 in human breast cancer specimens. It supports the emerging roles of tumor-infiltrating sympathetic and parasympathetic nerves in the anti-tumor immune response, consistent with a recent report that a β-adrenergic blocker decreased the expression of PD-1 and FOXP3 on lymphocytes in a mouse tumor model. In another embodiment, the present invention may be associated with the communication between the sympathetic nervous and immune systems reported in non-tumor settings; sympathetic nerves regulate the effecter function of CD8⁺ T cells, and egress of lymphocytes from lymph nodes, cell surface expression of molecules, and cytokine production by the expression of β-adrenergic receptors on lymphocytes. Together, tumor-infiltrating sympathetic nerves attenuate the anti-tumor immune response, whereas parasympathetic nerves enhance this response.

The genetic deletion of tumor-infiltrating local sympathetic nerves had greater tumor suppressing efficacy than the administration of α- and β-adrenergic receptor blockers in several models. This is not likely due to an insufficient drug dose, as the dose of phentolamine or propranolol used in the present study was set to be equivalent to or greater than that in previous arts. Next, because genetic neuroengineering is localized and selective for tumor tissue, systemic side effects, which are often observed in pharmacological treatments, are avoided. For example, immune checkpoint inhibitors are able to suppress tumor behavior, but can concurrently elicit deleterious autoimmunity. In contrast, local genetic neuroengineering can be applied to suppress immune checkpoints selectively in tumor tissue without eliciting systemic side effects such as autoimmunity. Moreover, genetic neuroengineering of tumor-infiltrating autonomic nerves can be individualized for the treatment of different types of cancers. Recent clinical studies suggest that the administration of β-blockers benefits patients with prostate or breast cancer, but not those with colorectal cancer or melanoma. As the vast majority of studies of beta-blockade are retrospective, there could be many reasons responsible for the differential impact of beta-blockers observed in these studies (e.g., characteristics of the population, length of treatment). However, these clinical studies suggest that therapies must be individualized for different cancer types. In addition, our findings in breast cancer differ from those in a recent report showing that parasympathetic cholinergic fibers promote cancer dissemination in prostate cancer through muscarinic 1 cholinergic mechanism and gastric cancer through muscarinic 3 cholinergic mechanism. Accordingly, advanced techniques to selectively manipulate local neural input for therapeutic purposes are desired and envisioned. The local genetic neuroengineering technique in the present invention meets the need for an individualizable therapeutic approach to different cancer types by allowing for the control of neurostimulation or denervation.

For deletion of tumor-infiltrating sympathetic nerves, AAV vectors carrying the diphtheria toxin A subunit (DTA), a strong lethal molecule, were injected downstream of the TH promoter into 50-mm³ tumors, which led to the loss of tumor-infiltrating TH⁺ sympathetic nerves; decreased tumor tissue NE content; and suppressed primary tumor growth and distant metastasis. These intratumoral injections of vectors did not affect the tissue NE content in normal organs (e.g., heart, kidney, and lower-limb skeletal muscle), indicating the tumor specificity of the neuroengineered invention.

The present invention may be used in specified combination with conventional chemotherapeutic oncologic agents and radiological treatments. And, likewise in combination with other medical instruments (such as employing ultra-sound to further locally stimulate parasympathetic nerves) or other pharmacological agents which beneficially modulate the autonomic nervous system with respect to specific tumor types. Such agents may include such as GABA, L-theanine, Hypericum perforatum, and Valeriana officinalis.

Viral Vector Preparation

Serotype 2 AAV vectors of AAV-TH-NaChBac T220A-2A-GCaMP3, AAV-TH-GCaMP3, AAV-TH-NaChBac T220A, AAV-TH-DTA, AAV-TH-CreERT, and AAV-TH-RFP were generated by techniques known in the arts (Kinoshita, M., et al. Genetic dissection of the circuit for hand dexterity in primates. Nature 487, 235-238 (2012)). A 2.5-kb rat TH promoter was obtained from rat genomic DNA (Clontech, Mountain View, Calif.) by PCR (forward primer, 5′-GGCCTAAGAGGCCTCTTGGGAT-3′; reverse primer, 5′-CTGGTGGTCCCGAGTTCTGTCT-3′) and confirmed by DNA sequence analysis. CreERT was made from pCAG-CreERT2 (Plasmid #14797, Addgene, Cambridge, Mass.) and ligated downstream of the TH promoter based on pAAV-MCS (Agilent Technologies, Santa Clara, Calif.). A fragment containing DTA was obtained from DTA PGKdtabpA (Plasmid #13440, Addgene). The RFP cDNA was purchased from Evrogen. In addition, serotype 2 AAV vectors of AAV-Floxed-EGFP-eTeNT, AAV-Floxed-DTA, AAV-Floxed-NaChBac T220A-2A-GCaMP3, AAV-Floxed-GCaMP3, AAV-Floxed-NaChBac T220A, AAV-ChAT-NaChBac T220A-2A-GCaMP3, AAV-ChAT-GCaMP3, AAV-ChAT-Cre, and lentiviral vector of LV-TRE-EGFP-eTeNT were generated by techniques known in the arts. These vectors contained the woodchuck hepatitis virus post-transcriptional regulatory element sequence and the SV40 polyadenylation signal sequence of the pCMV script vector.

### EXAMPLES

Deleting Tumor-Infiltrating Sympathetic Nerves Suppresses the Expression of Immune Checkpoint Molecules in the Tumor Microenvironment of Human Breast Cancer Cell Xenografts

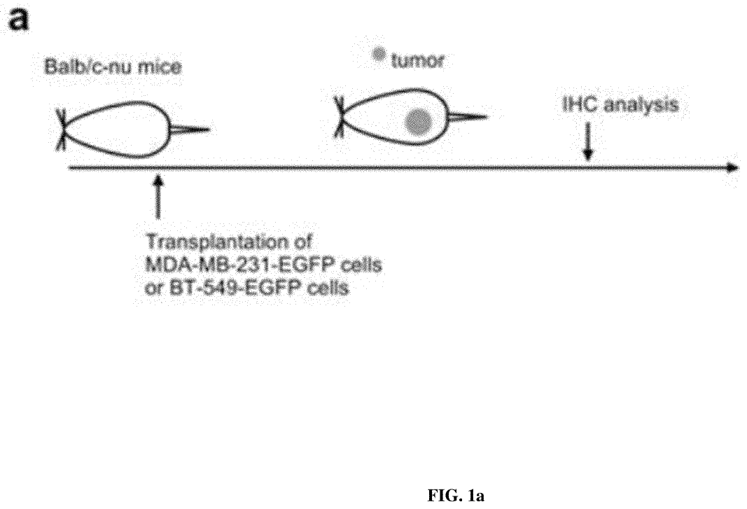

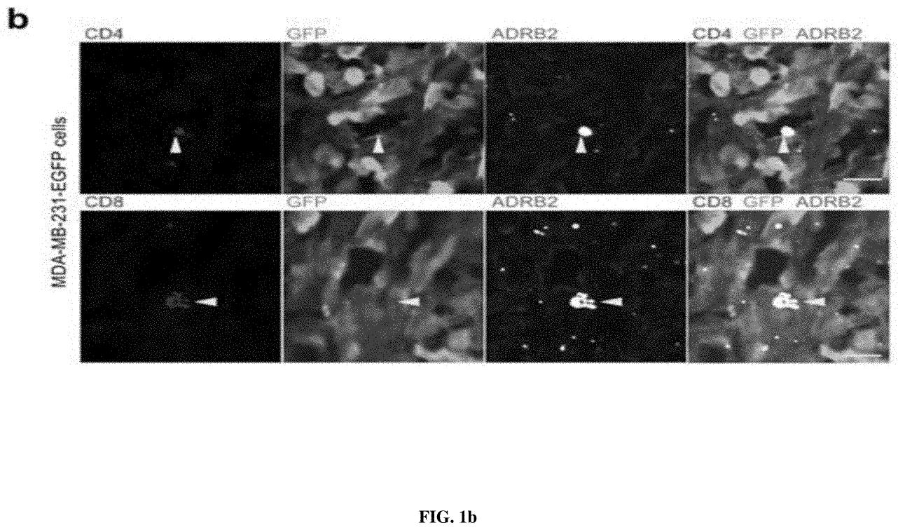

The mouse xenograft model of human breast cancer is examined whether it has CD4⁺ and CD8⁺ tumor-infiltrating lymphocytes (TIL) as shown in FIG. 1a. While FIG. 1b shows immunofluorescence staining of xenograft tumor revealing a presence of CD4⁺ and CD8⁺ TIL in tumor microenvironment of these Balb/c-nu mice. Moreover, these CD4⁺ and CD8⁺ TILs expressed β2-adrenergic receptors, with approximately 30% and 70% of expression in CD4⁺ and CD8⁺ T cells, respectively as shown in FIG. 1c.

Next, programmed death-1 (PD-1), programmed death ligand-1 (PD-L1), and FOXP3 are immune checkpoint molecules that lead to immunosuppression in the tumor microenvironment.

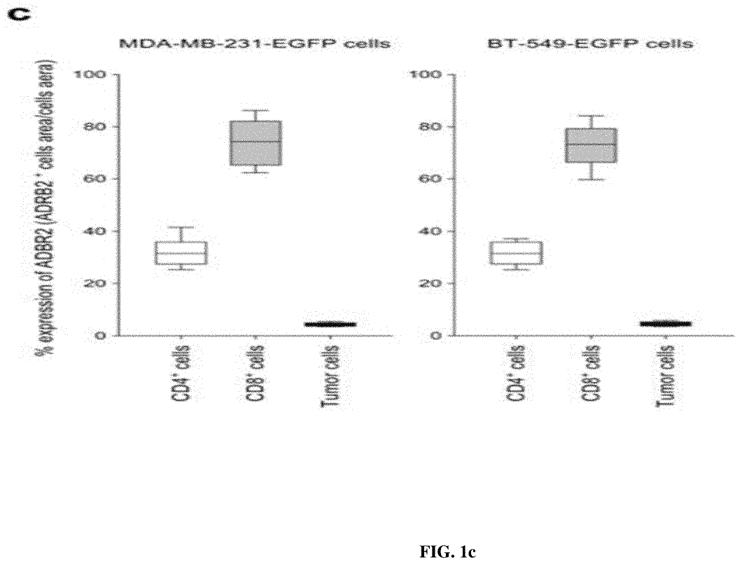

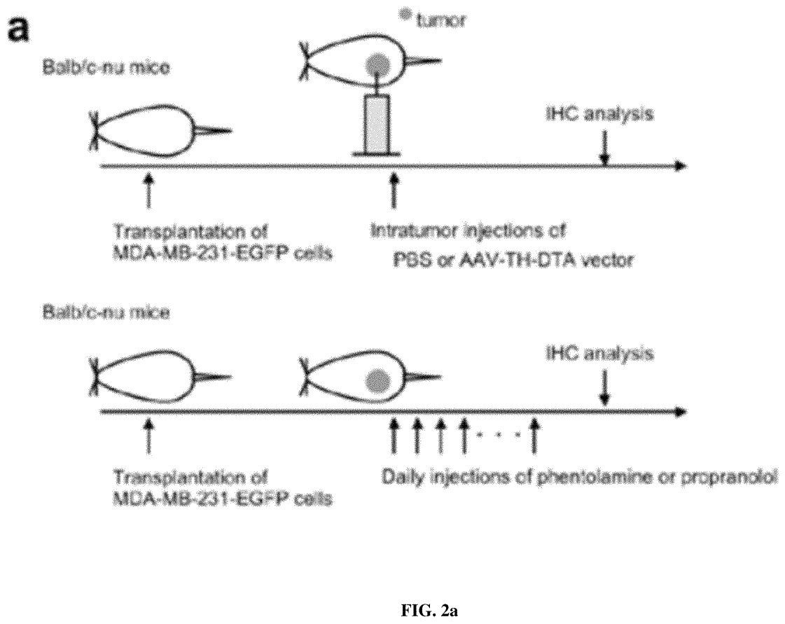

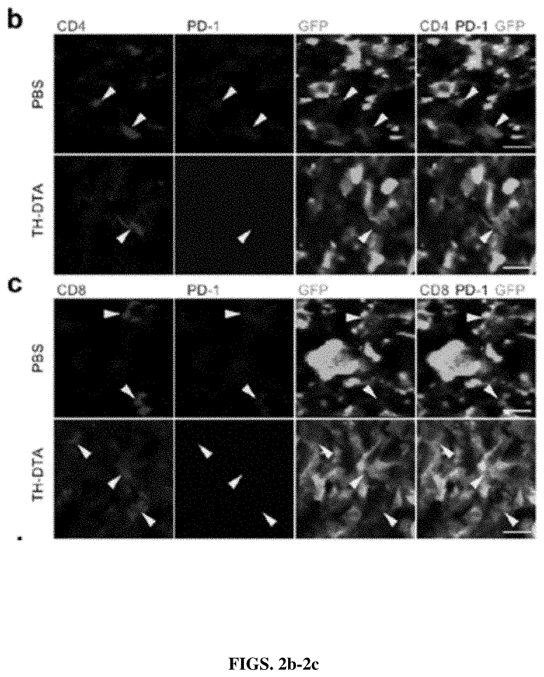

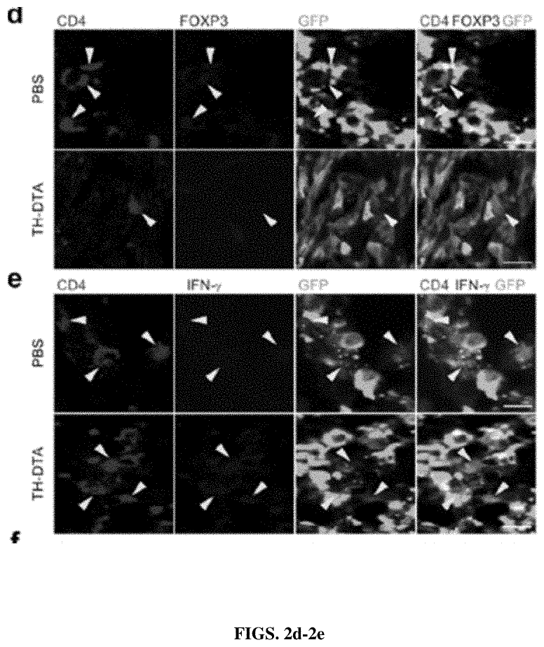

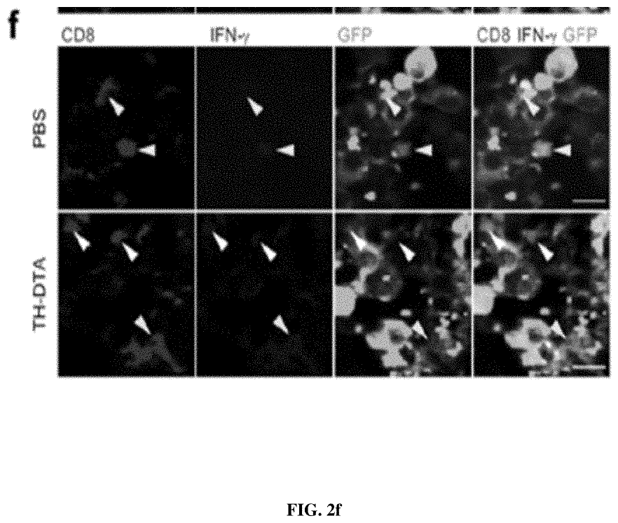

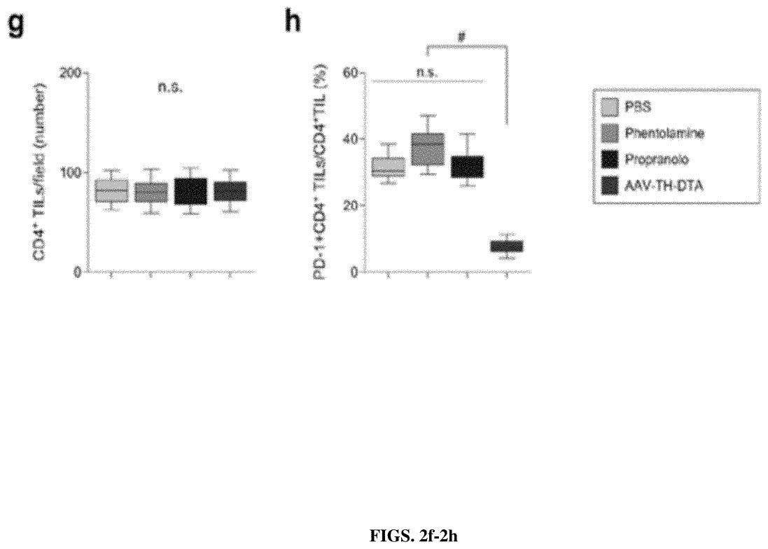

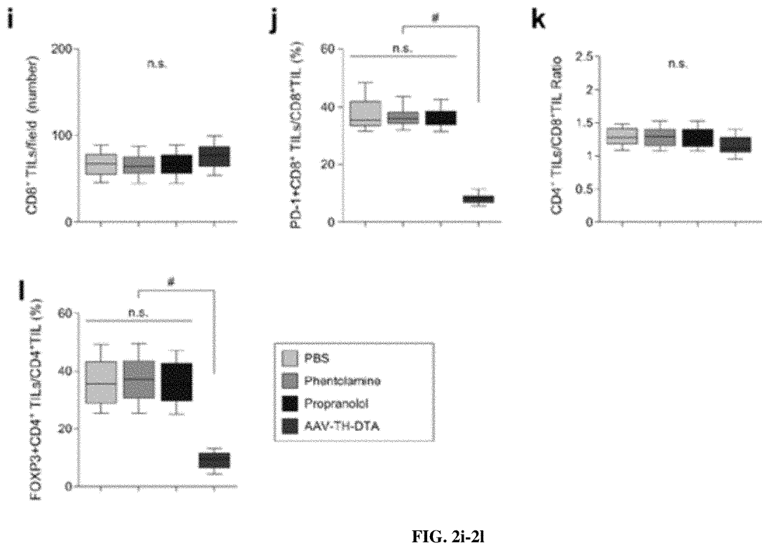

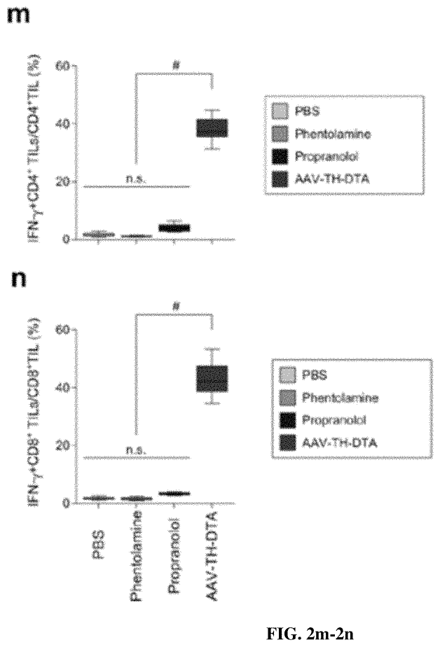

FIG. 2a examines whether the genetic deletion of tumor-infiltrating sympathetic nerves alters the expression of these immune checkpoint molecules in the tumor microenvironment.

Immunofluorescence staining of xenograft tumors of Balb/c-nu mice had genetic sympathetic denervation by injecting the AAV-TH-DTA vector into 50-mm³ tumors unaltered the number of CD4⁺ as shown in FIG. 2g and suppressed expression of PD-1 on CD4⁺ TIL, as shown in FIG. 2b, and FIG. 2h. Genetic sympathetic denervation unaltered the number of CD8⁺ TIL as shown in FIG. 2i and suppressed expression of PD-1 on CD8⁺ TIL as shown in FIG. 2c, and FIG. 2j. FIG. 2k shows genetic sympathetic denervation unaltered the CD4+/CD8⁺ TIL ratio. Genetic sympathetic denervation suppressed expression of Foxp3 on CD4⁺ TIL as shown in FIG. 2d and FIG. 2l. Next, interferon-gamma (IFN-γ) is a cytokine critical to adaptive anti-tumor immunity in CD4⁺ and CD8⁺ T cells. The genetic sympathetic denervation by AAV-TH-DTA vector increased expression of IFN-γ both on CD4⁺ as shown in FIG. 2e and FIG. 2m and CD8⁺ TILs as shown in FIG. 2f and FIG. 2n. These effects were greater than those by daily injections of phentolamine or propranolol, which did not alter immunological parameters as shown in FIG. 2g to FIG. 2n which compare PBS vs. Phentolamine and PBS vs. Propranolol. The limited efficacy of propranolol injections may partly be explained by arterial baroreflex mechanism, since addition of bilateral SAD to daily injections of propranolol led to following significant effects: it led to lower expressions of PD-1 on CD4⁺ TIL, comparing propranolol+sham vs. propranolol+SAD, PD-1 on CD8⁺ TIL and Foxp3 on CD4⁺ TIL and greater expressions of IFN-γ both on CD4⁺ and CD8⁺ TILs. Together, these results suggest that deletion of tumor-infiltrating local sympathetic nerves suppresses the expression of immune checkpoint molecules in the tumor microenvironment.

Deleting Tumor-Infiltrating Sympathetic Nerves Suppresses the Expression of Immune Checkpoint Molecules in the Tumor Microenvironment of Chemically-Induced Breast Cancer

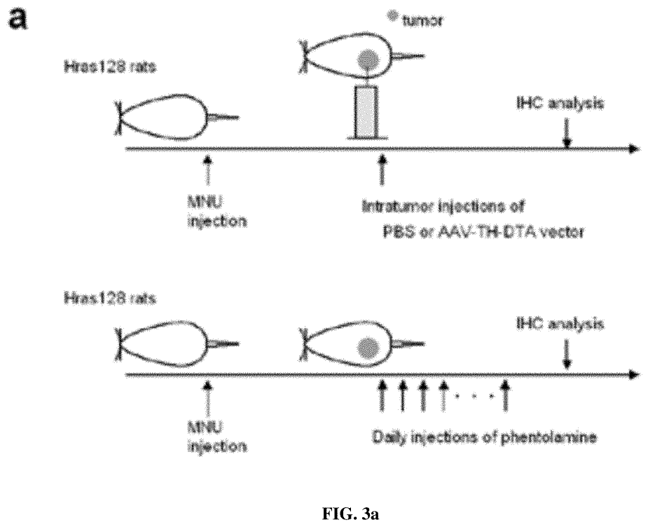

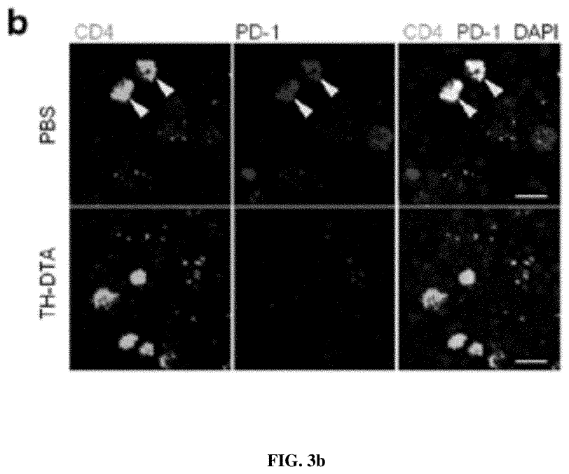

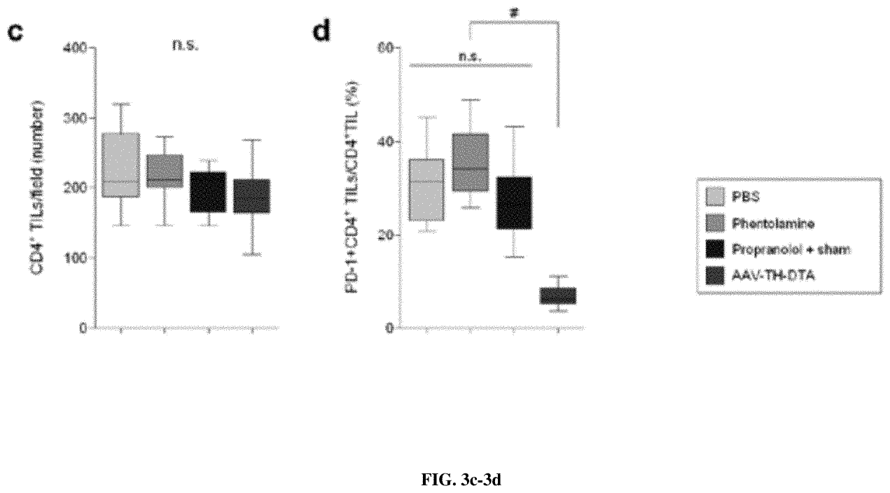

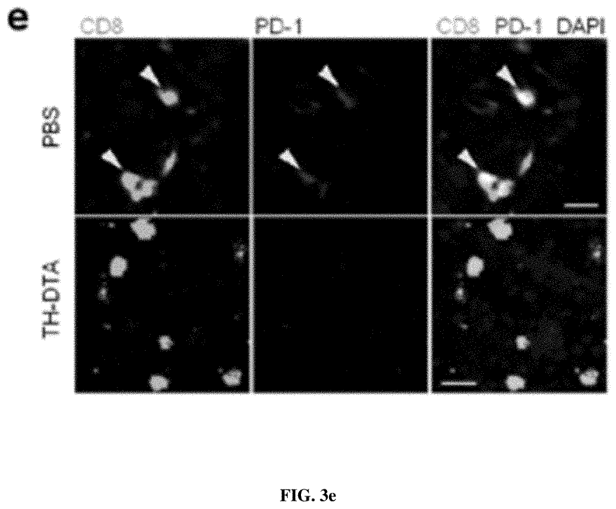

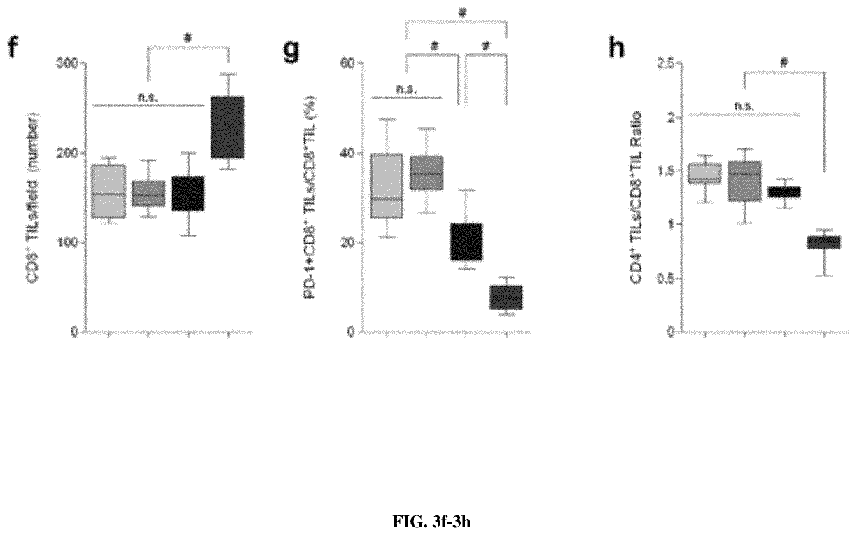

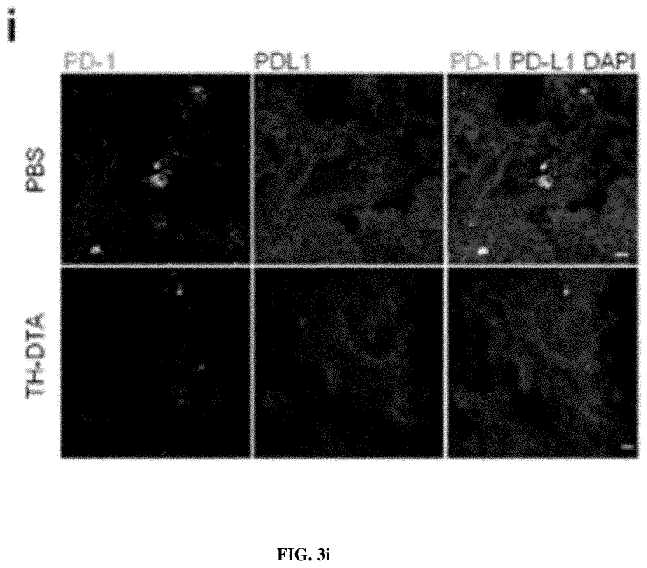

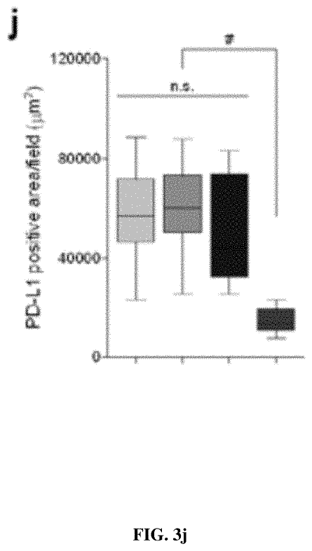

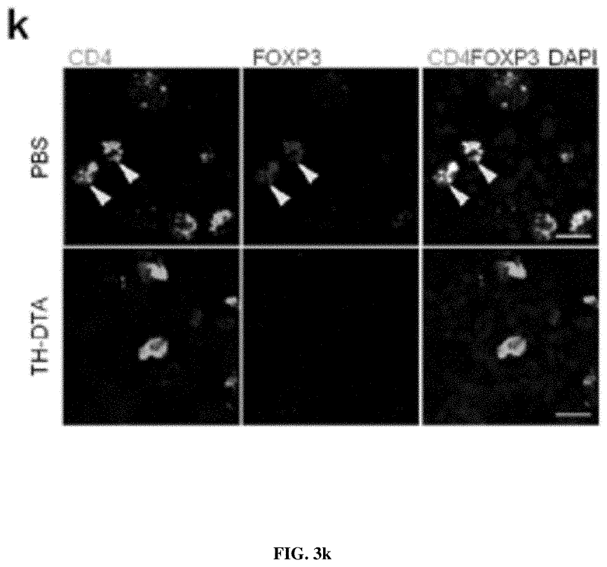

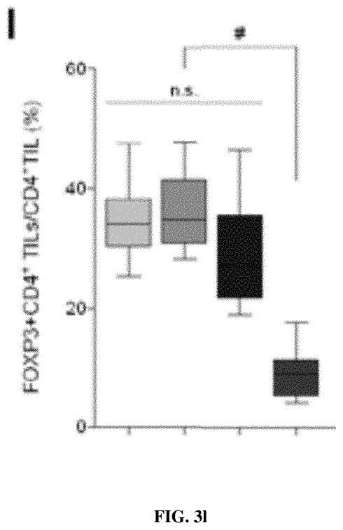

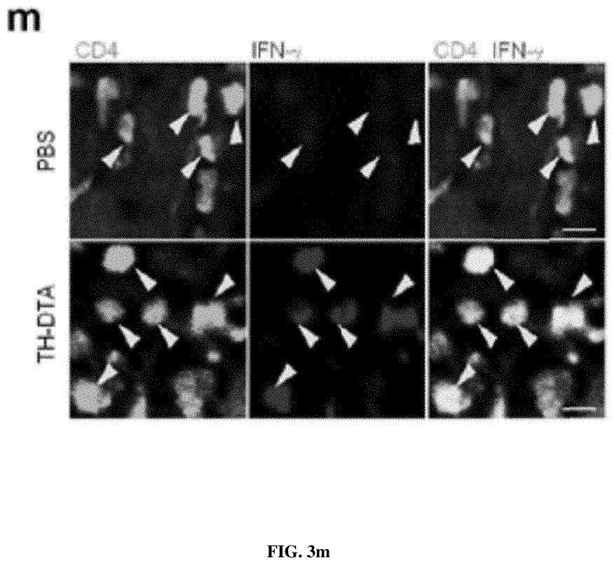

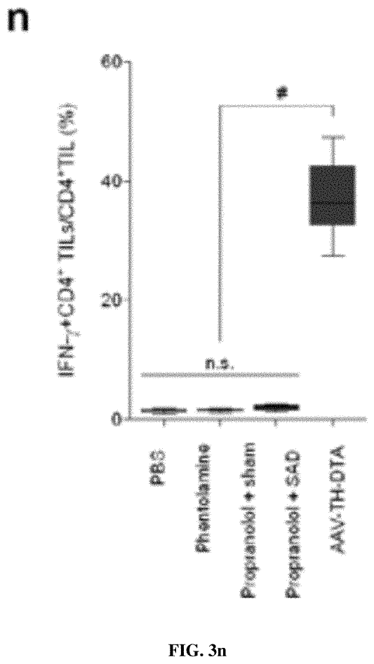

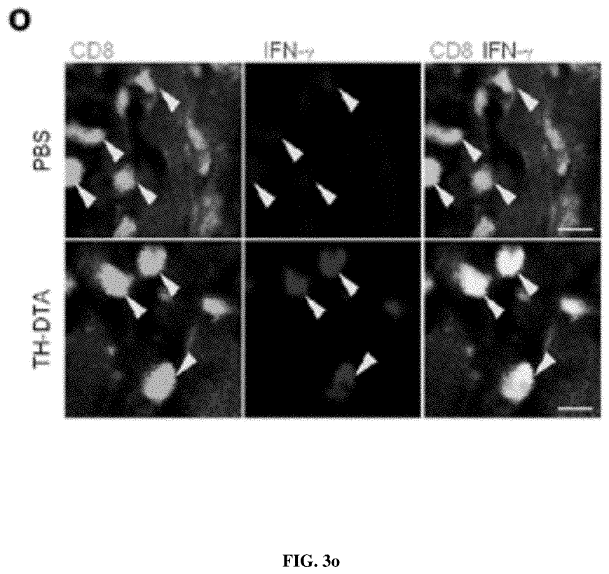

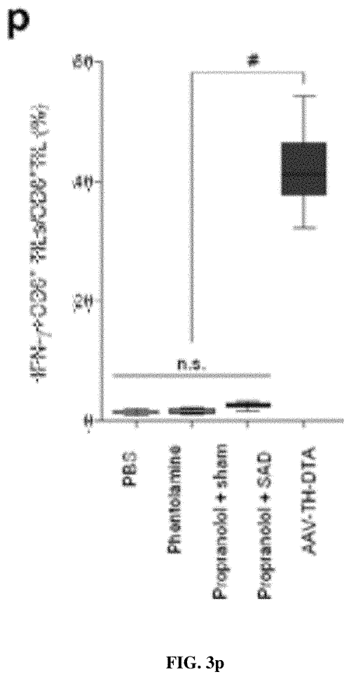

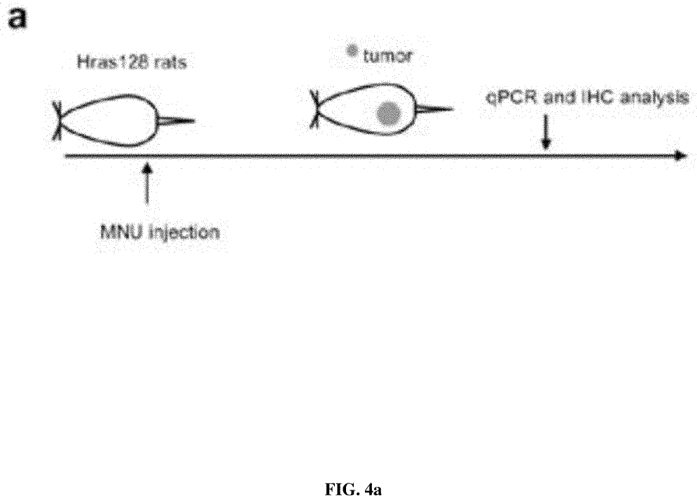

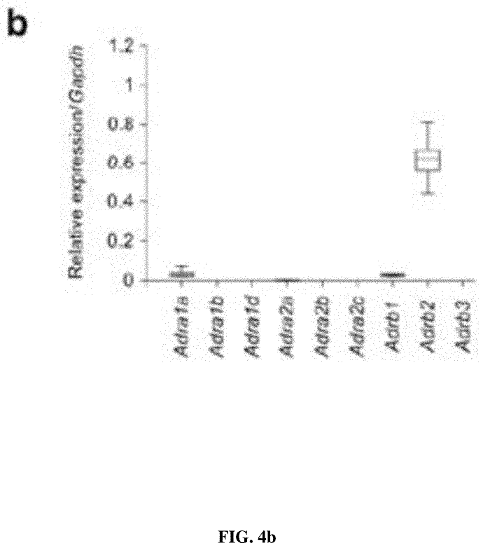

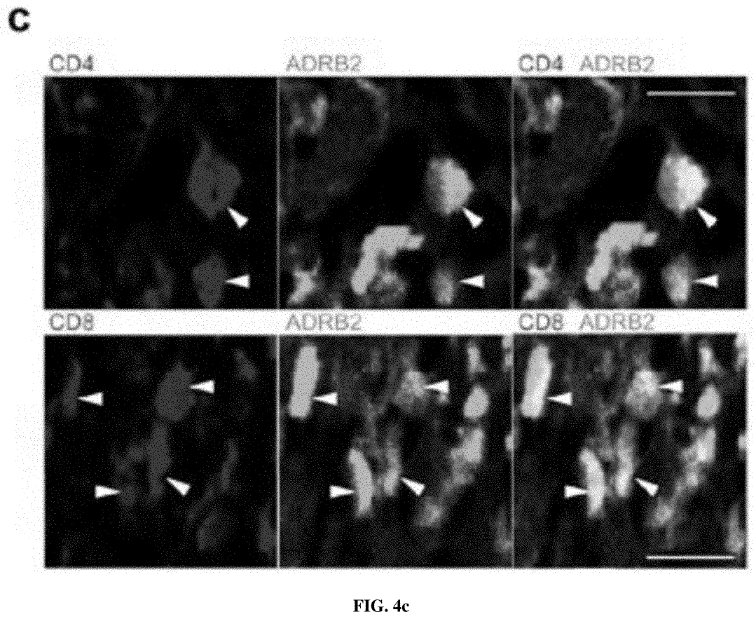

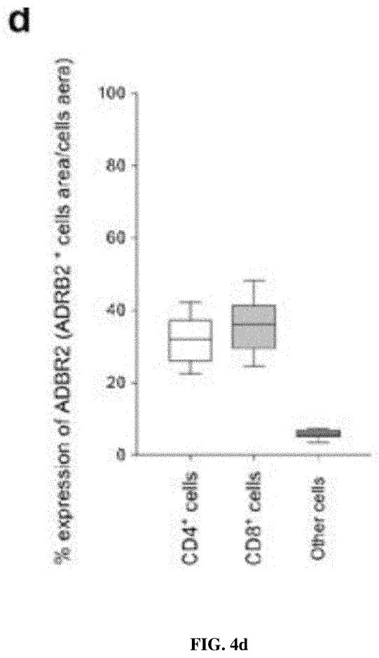

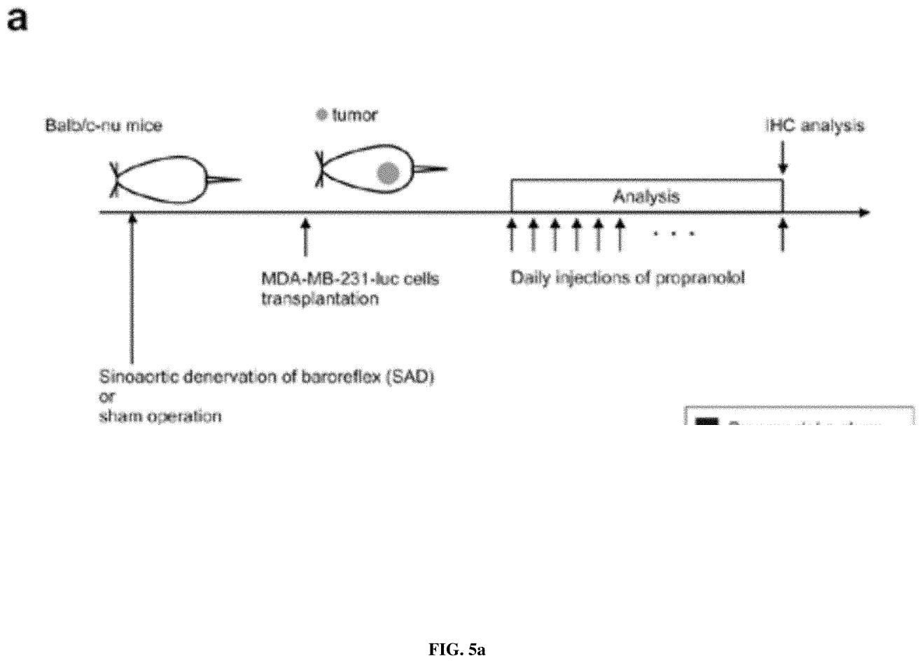

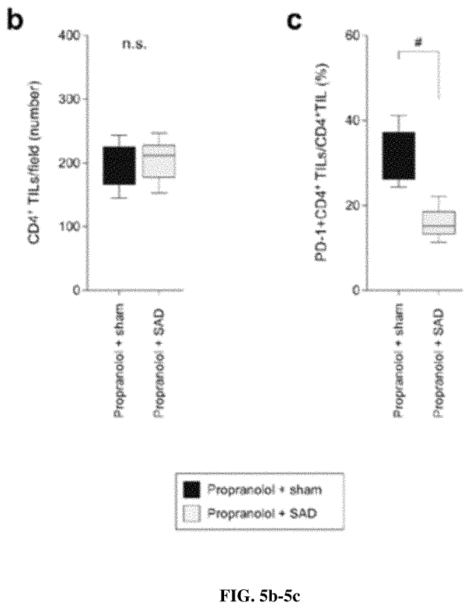

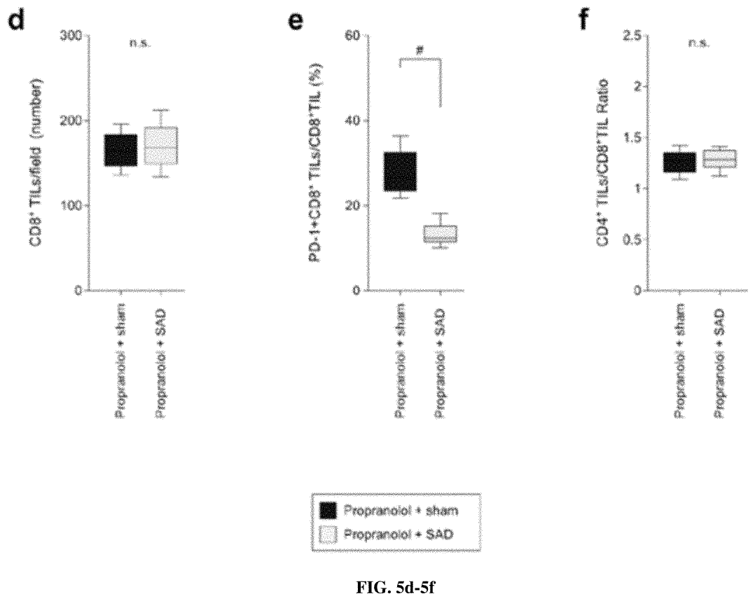

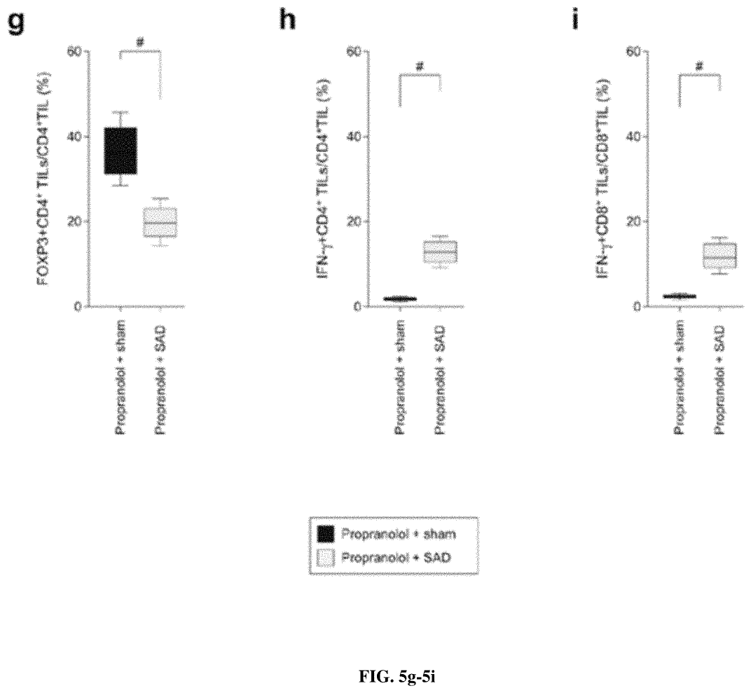

The genetic deletion of tumor-infiltrating sympathetic nerves is examined whether it alters the expression of these immune checkpoint molecules in the tumor microenvironment of chemically-induced breast cancer as shown in FIG. 3a. Immunofluorescence staining of MNU-induced breast tumors in Hras128 rats showed that genetic sympathetic denervation by injecting the AAV-TH-DTA vector into 10³-mm³ tumors unaltered the number of CD4⁺ TIL as shown in FIG. 3c and suppressed expression of PD-1 on CD4⁺ TIL as shown in FIG. 3b and FIG. 3d. Genetic sympathetic denervation increased the number of CD8⁺ TIL as shown in FIG. 3f and decreased expression of PD-1 on CD8⁺ TIL as shown in FIG. 3e and FIG. 3g. As shown in FIG. 3h, genetic sympathetic denervation suppressed the CD4+/CD8⁺ TIL ratio, an indicator of a poor clinical outcome. Genetic sympathetic denervation suppressed expression of PD-L1 on the tumor tissue as shown in FIG. 3i and FIG. 3j and that of Foxp3 on CD4⁺ TIL as shown in FIG. 3k and FIG. 3l. These effects were greater than those induced by daily injections of phentolamine or propranolol, which did not alter these parameters as shown FIG. 3d to FIG. 3h, FIG. 3j and FIG. 3l except for a slight decrease in the expression of PD-1 on CD8⁺ TIL by propranolol as shown in FIG. 3g. In addition, genetic sympathetic denervation by AAV-TH-DTA vector increased expressions of IFN-γ on CD4⁺ TIL as shown in FIG. 3m and FIG. 3n and CD8⁺ TIL as shown in FIG. 3o and FIG. 3p. Whereas daily injections of phentolamine or propranolol unaltered them. Immunofluorescence analysis showed that CD4⁺ and CD8⁺ T cells in MNU-induced breast tumors expressed β2-adrenergic receptors as shown in FIG. 4c with approximately 30-40% of expression as shown in FIG. 4d. The limited effects of propranolol injections on immuno-related parameters may partly be explained by baroreflex mechanism, since addition of bilateral SAD to daily injections of propranolol led to lower expressions of PD-1 on CD4⁺ TIL as shown in FIG. 5c, comparing propranolol+sham vs. propranolol+SAD and PD-1 on CD8⁺ TIL as shown in FIG. 5e and Foxp3 on CD4⁺ TIL as shown in FIG. 5g and greater expressions of IFN-γ both on CD4⁺ TIL as shown in FIG. 5h and CD8⁺ TIL as shown in FIG. 5i. Together, these results suggest that deletion of tumor-infiltrating local sympathetic nerves suppresses the expression of immune checkpoint molecules in the tumor microenvironment.

Stimulating Tumor-Infiltrating Parasympathetic Nerves can Suppress the Expression of Immune Checkpoint Molecules in the Tumor Microenvironment of Human Breast Cancer Cell Xenografts

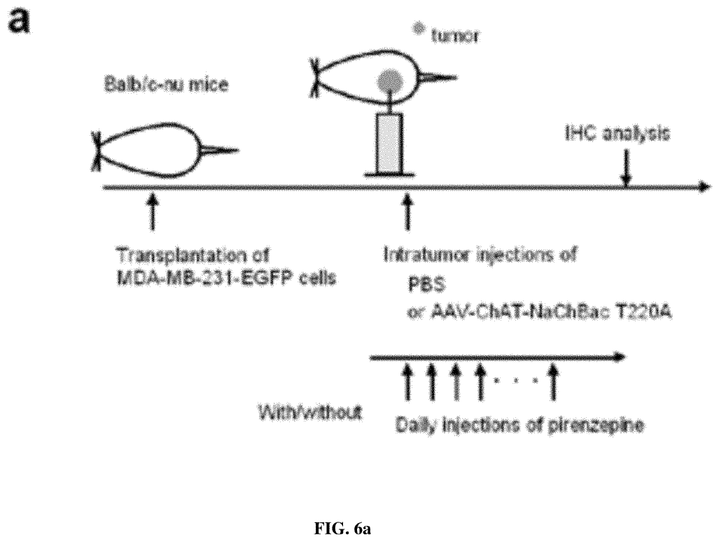

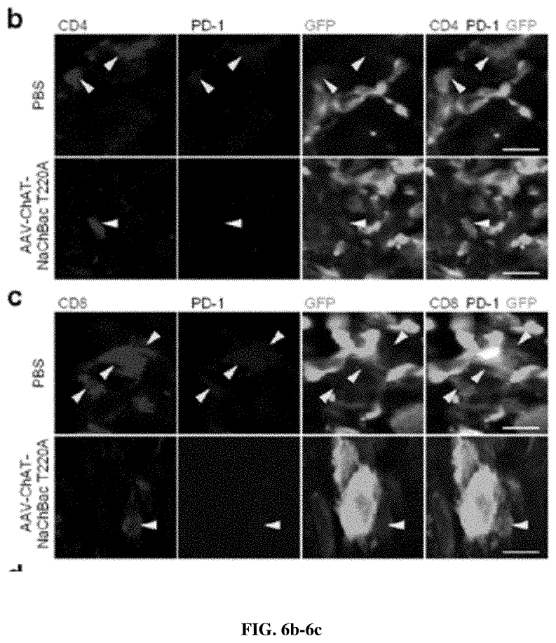

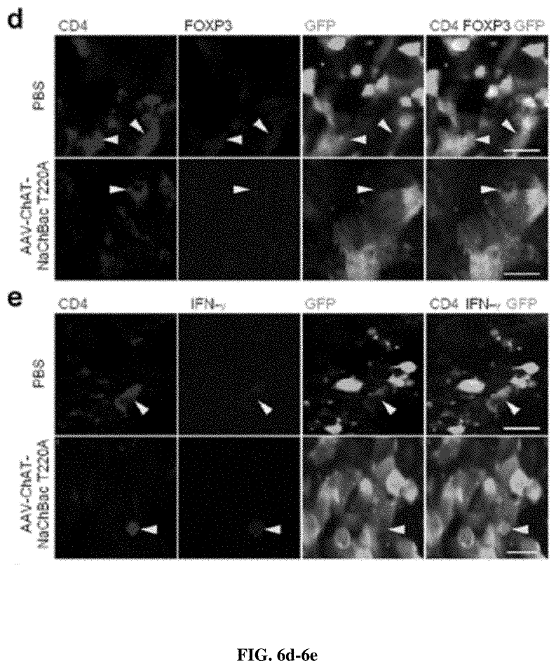

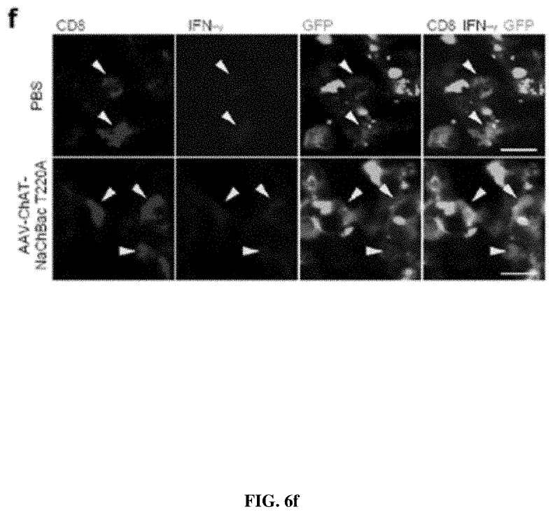

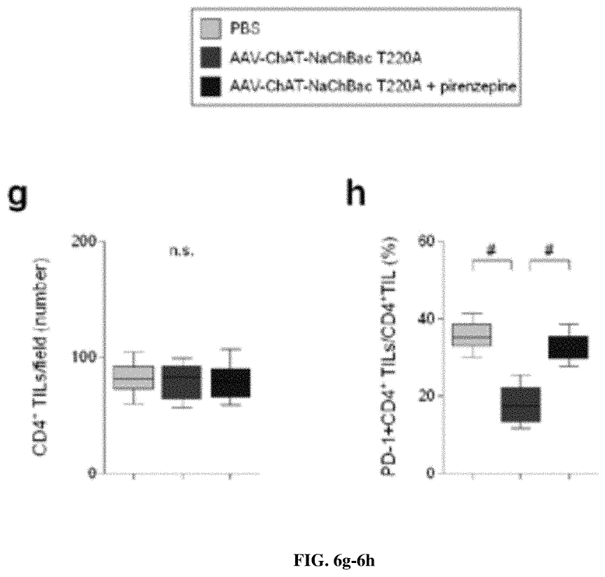

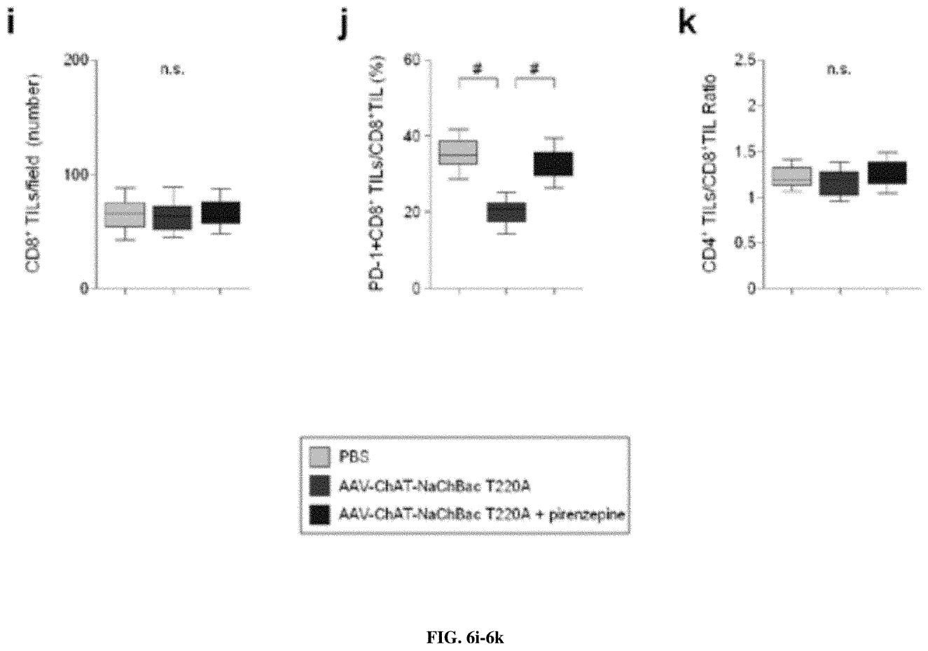

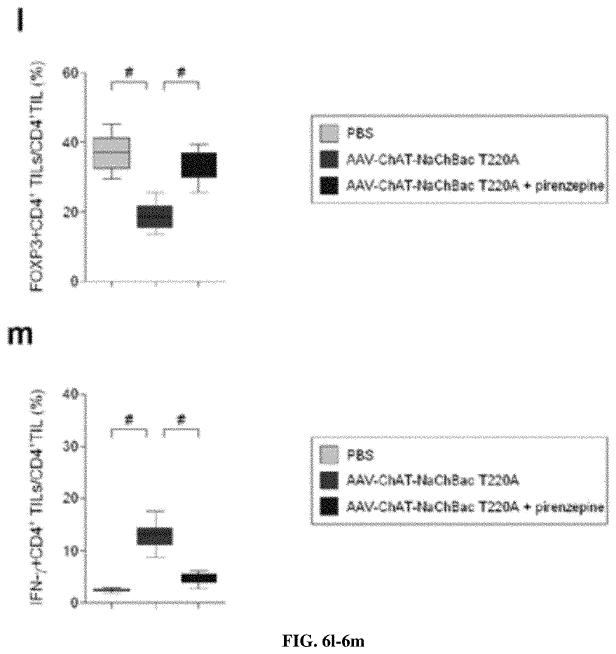

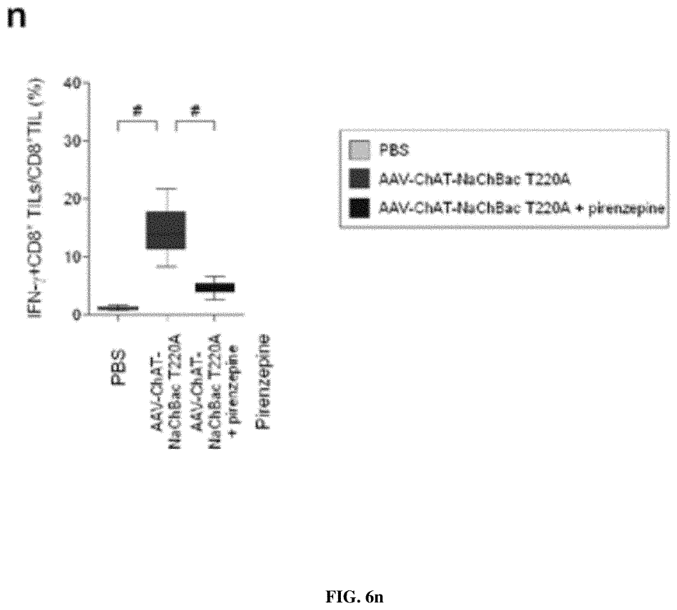

As shown in FIG. 6a to FIG. 6n, the genetic neurostimulation of tumor-infiltrating parasympathetic nerves altered the expression of immune checkpoint molecules in the tumor microenvironment of MDA-MB-231 human breast cancer xenografts. Parasympathetic neurostimulation by injecting AAV-ChAT-NaChBac T220A vector into 50-mm³ tumors unaltered the number of CD4⁺ TIL as shown in FIG. 6g and decreased expression of PD-1 on CD4⁺ TIL as shown in FIG. 6b and FIG. 6h. Parasympathetic neurostimulation unaltered the number of CD8⁺ TIL as shown in FIG. 6i and decreased expression of PD-1 on CD8⁺ TIL as shown in FIG. 6c and FIG. 6j. Parasympathetic neurostimulation unaltered the ratio of CD4+/CD8⁺ TIL as shown in FIG. 6k. Parasympathetic neurostimulation decreased expression of Foxp3 on CD4⁺ TIL as shown in FIG. 6d and FIG. 6l. In addition, parasympathetic neurostimulation increased expression of IFN-γ both on CD4⁺ TIL as shown in FIG. 6e and FIG. 6m and CD8⁺ TIL as shown in FIG. 6f and FIG. 6n. These immuno-modulation effects by parasympathetic neurostimulation were inhibited by daily injections of pirenzepine as shown in FIG. 6h, FIG. 6j, FIG. 6l to FIG. 6m, suggesting involvement of CHRM1 mechanism. These results suggest that neurostimulation of tumor-infiltrating parasympathetic nerves suppresses expressions of immune checkpoint molecules in the tumor microenvironment of mouse xenograft model of human breast cancer.

Stimulating Tumor-Infiltrating Parasympathetic Nerves can Suppress the Expression of Immune Checkpoint Molecules in the Tumor Microenvironment of Chemically-Induced Breast Cancer

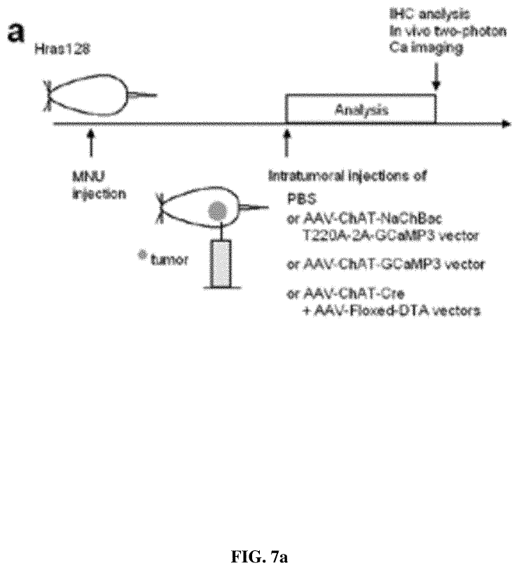

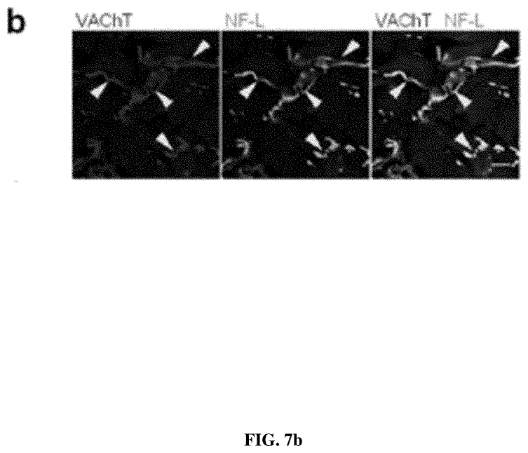

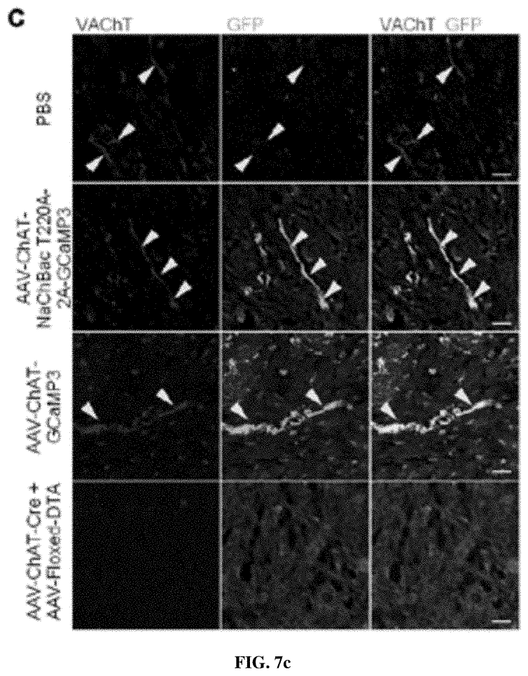

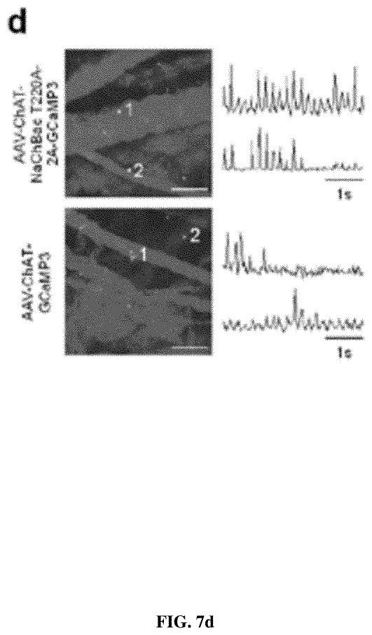

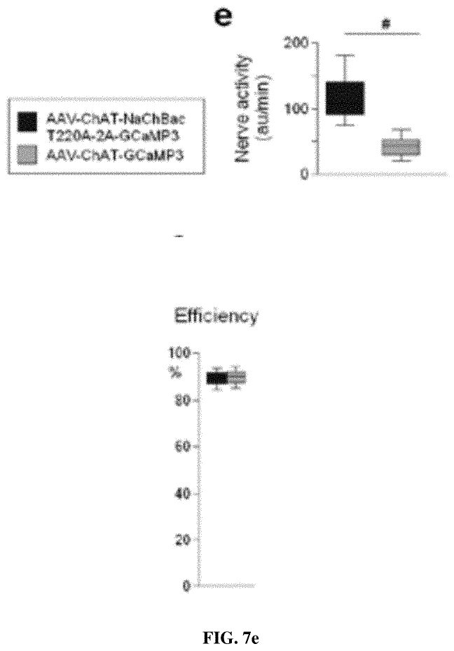

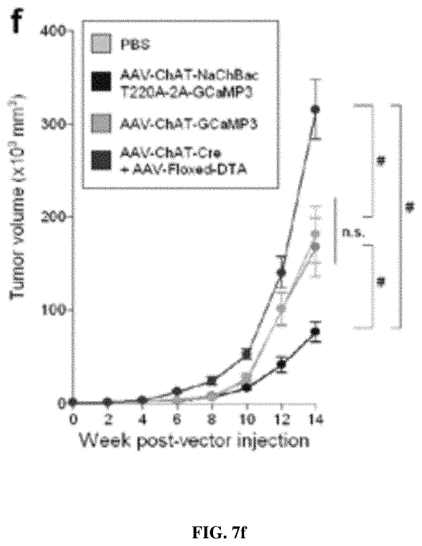

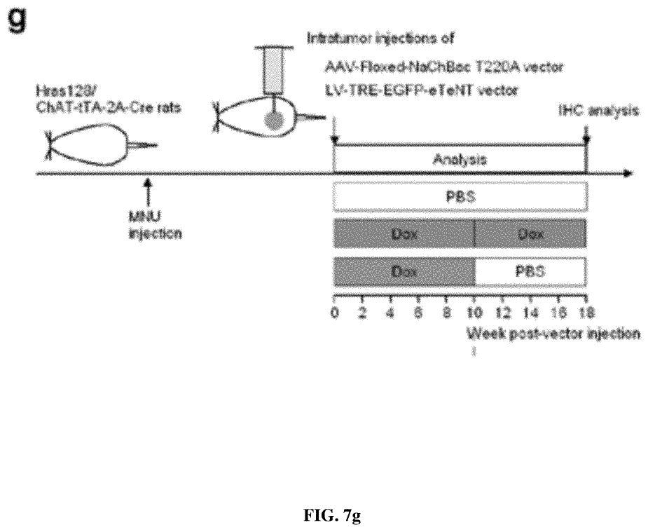

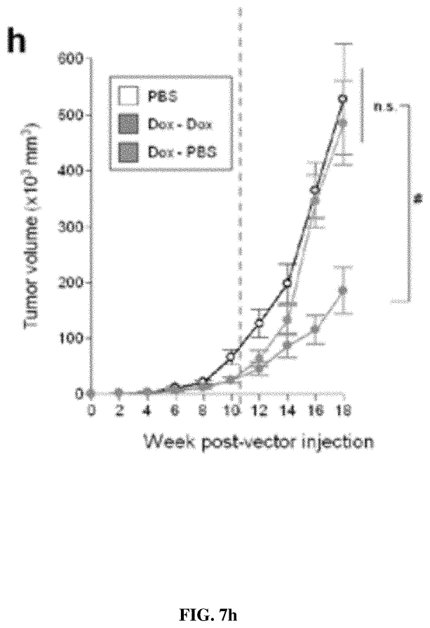

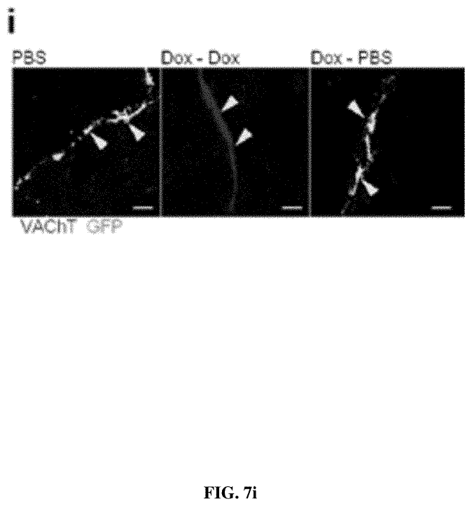

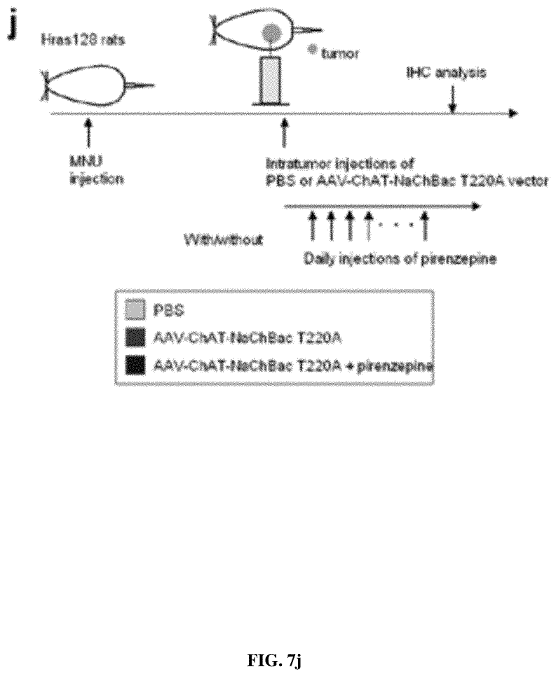

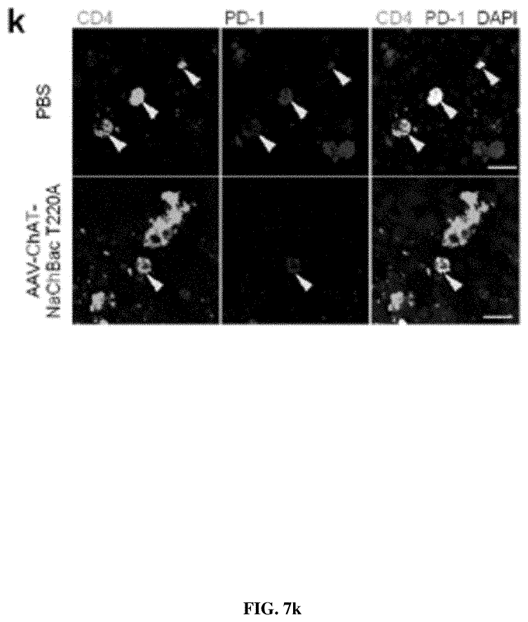

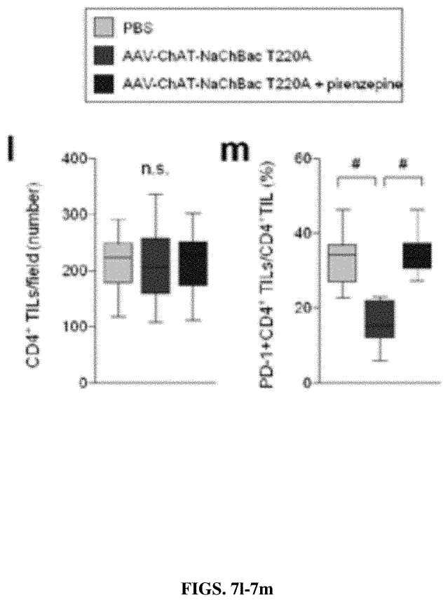

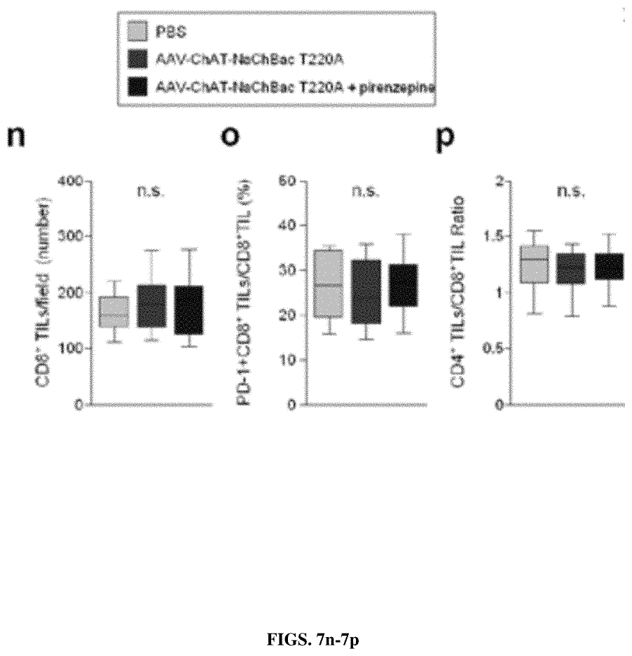

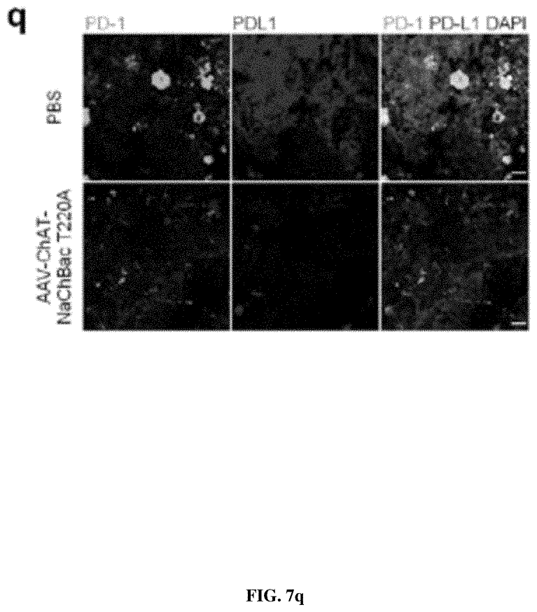

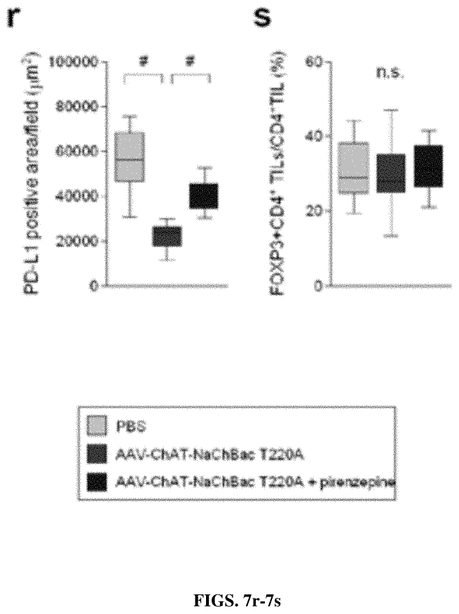

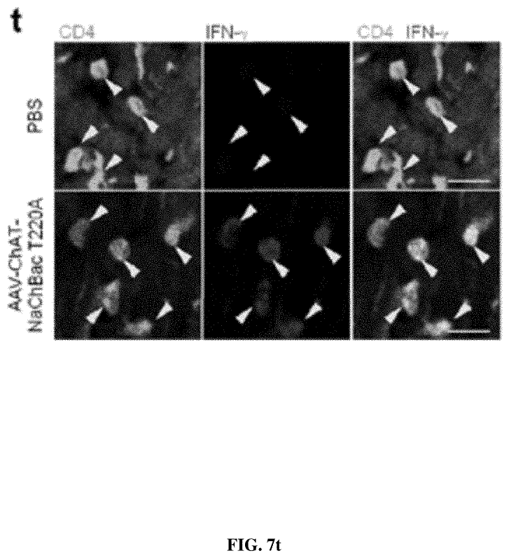

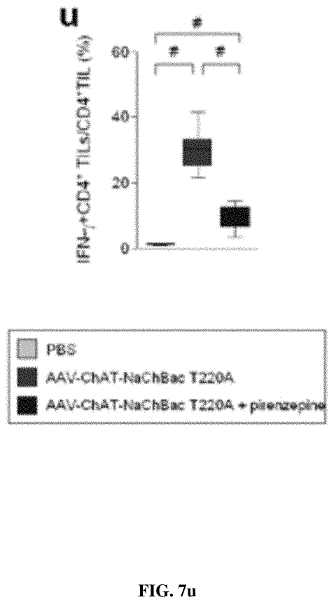

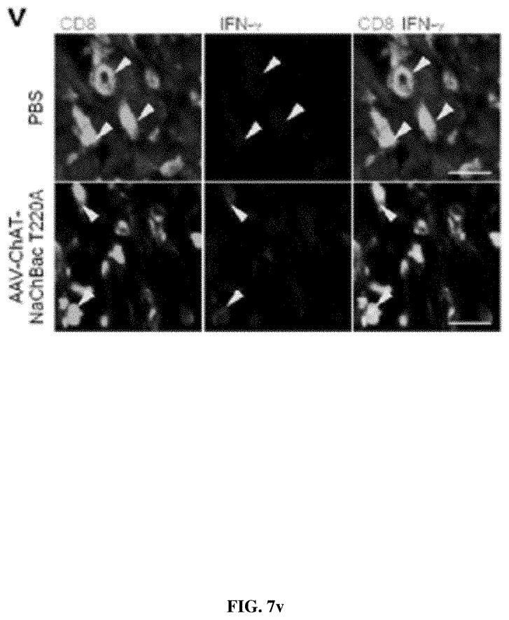

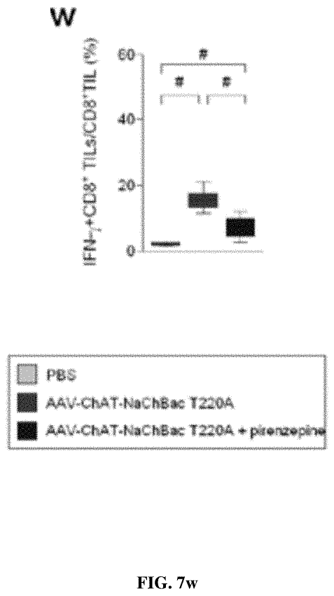

As shown in FIG. 7j, genetic neurostimulation of tumor-infiltrating parasympathetic nerves alters the expression of immune checkpoint molecules in the tumor microenvironment. MNU-induced breast tumors of female Hras128 rats was immunofluorescently stained. Parasympathetic neurostimulation by injecting the AAV-ChAT-NaChBac T220A vector into 103-mm³ tumors unaltered the number of CD4⁺ as shown in FIG. 7l and decreased expression of PD-1 on CD4⁺ as shown in FIG. 7k and FIG. 7m. As shown in FIG. 7n, parasympathetic neurostimulation unaltered the number of CD8⁺ TIL and expression of PD-1 on CD8⁺ TIL as shown in FIG. 7o. Parasympathetic neurostimulation unaltered the ratio of CD4+/CD8⁺ TIL as shown in FIG. 7p. As shown in FIG. 7q and FIG. 7r, parasympathetic neurostimulation decreased expression of PD-L1 on the tumor tissue and unaltered expression of Foxp3 on CD4⁺ TIL as shown in FIG. 7s. In addition, as shown in FIG. 7t to FIG. 7w, parasympathetic neurostimulation increased expression of IFN-γ both on CD4⁺ and CD8⁺ TILs. These immuno-related effects by parasympathetic neurostimulation were inhibited by daily injections of pirenzepine as shown in FIG. 7m, FIG. 7r, FIG. 7u and FIG. 7w, suggesting involvement of M1 cholinergic receptor mechanism. These results suggest that stimulation of tumor-infiltrating parasympathetic nerves suppresses the expression of immune checkpoint molecules in the tumor microenvironment of chemically-induced breast cancer, but the effects are limited compared with genetic denervation of tumor-infiltrating sympathetic nerves.

Tumor-Infiltrating Autonomic Nerves in Human Breast Cancer

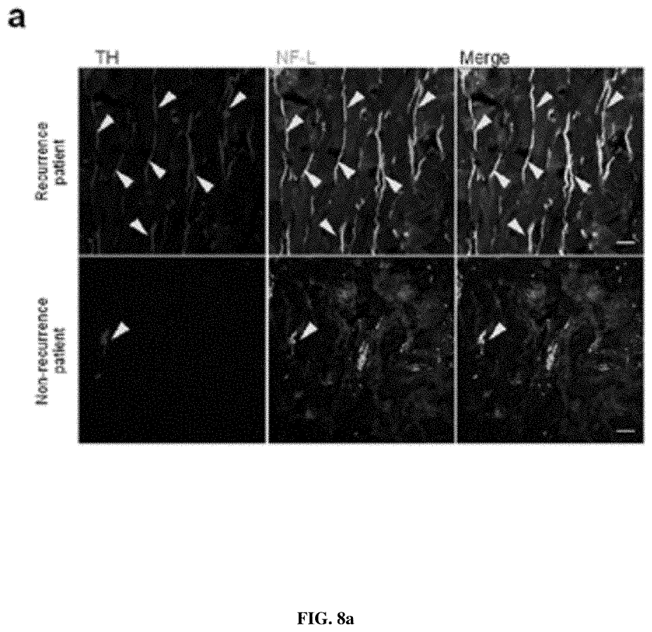

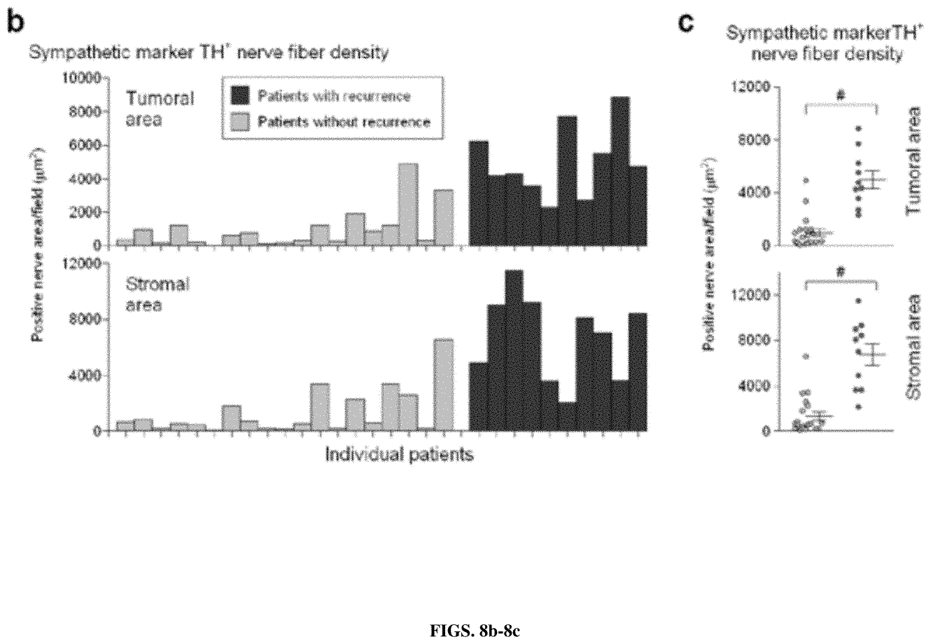

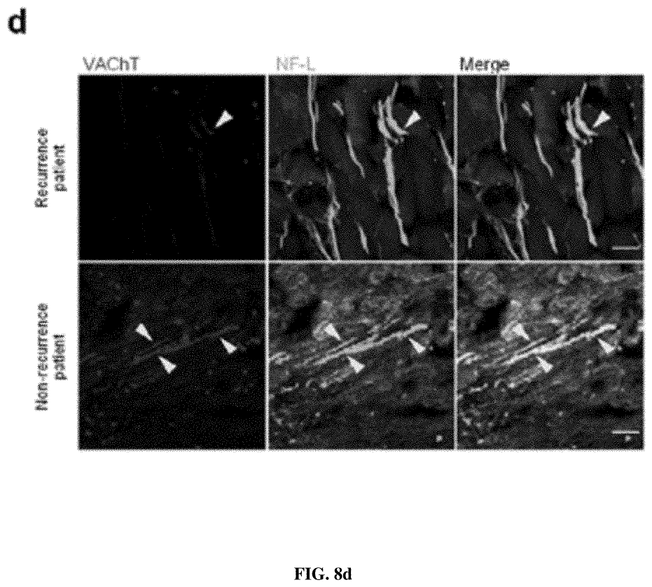

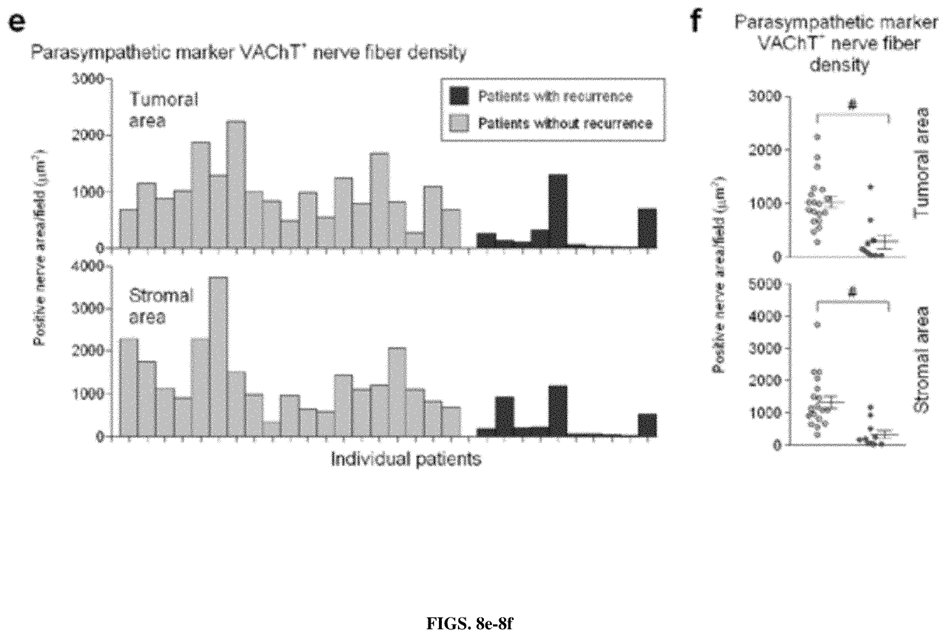

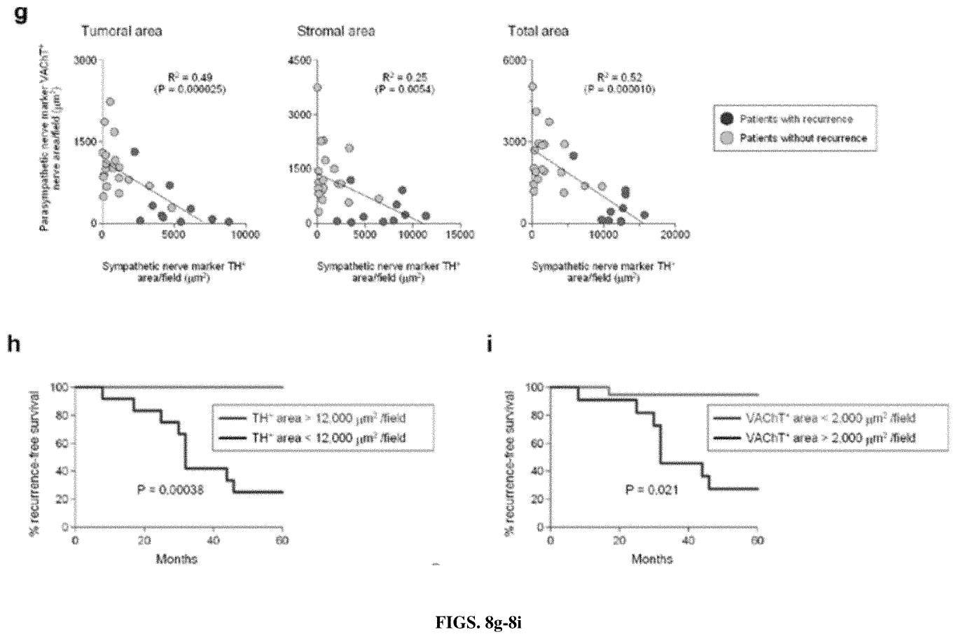

Although recent retrospective clinical studies reported the potential efficacy of β-blocker administration in patients with breast cancer, infiltration of human breast tumors by autonomic nerves has not yet been examined in detail. Accordingly, human breast cancer specimens were retrospectively analyzed as shown in FIG. 8. Twenty-nine patients underwent surgical resection of primary breast cancers at the National Cancer Center Hospital, Japan. Subjects were 10 patients with recurrence and 19 without. Autonomic nerve fiber densities were quantified by investigators blind to the clinical information. Immunofluorescence staining showed that TH⁺ NF-L⁺ sympathetic nerves as well as VAChT⁺ NF-L⁺ parasympathetic nerves infiltrated the tumor microenvironment as shown in FIG. 8a and FIG. 8d. TH⁺ NF-L⁺ sympathetic nerve fibers innervated the tumor/stromal area densely in patients with recurrence and sparsely in patients without as shown in FIG. 8a and FIG. 8b. Thus, the TH⁺ sympathetic nerve fiber density was greater in patients with recurrence than in those without as shown in FIG. 8c. In contrast, VAChT⁺ parasympathetic nerve fiber density in the tumor/stromal area was lower in patients with recurrence than in patients without as shown in FIG. 8d to FIG. 8f. Pooled data from all patients showed that TH⁺ sympathetic nerve fiber density negatively correlated with the VAChT⁺ parasympathetic nerve fiber density (tumoral, R²=0.49 P<0.01; stromal, R²=0.25 P<0.01; and total area, R²=0.45 P<0.01, as shown in FIG. 8g). These findings may relate to the small sample size that limits the possibility of detecting the independent prognostic value of the biomarkers. However, specific thresholds of TH⁺ nerve areas per field (>12,000 mm² per field; as shown in FIG. 8h) or VAChT⁺ nerve areas per field (<2,000 mm² per field; as shown in FIG. 8i) at diagnosis were associated with lower recurrence-free survival rates. These preliminary results suggest that human breast cancer is infiltrated by both sympathetic and parasympathetic nerve fibers, and that higher and lower densities of sympathetic and parasympathetic nerve fibers, respectively, are associated with a poor clinical outcome.

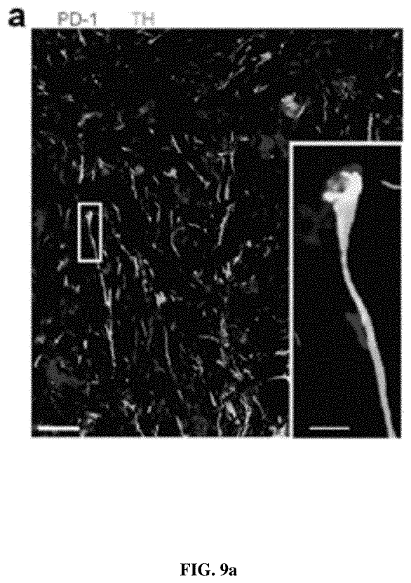

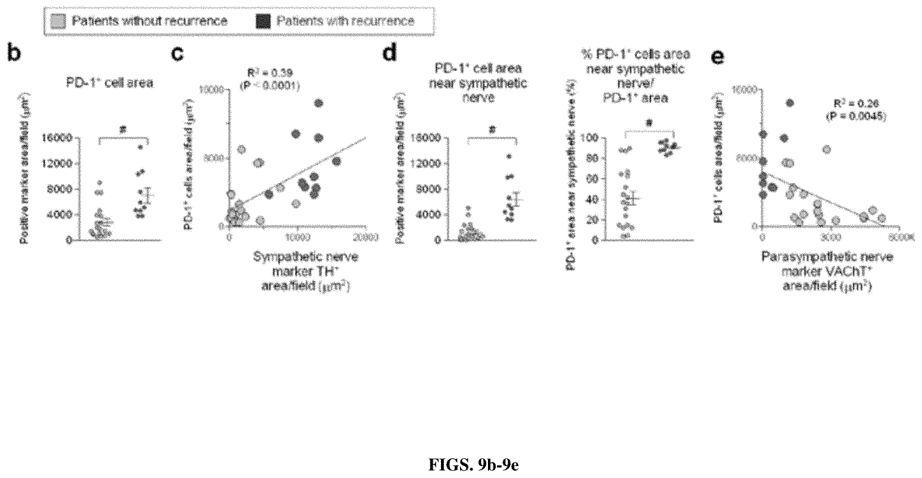

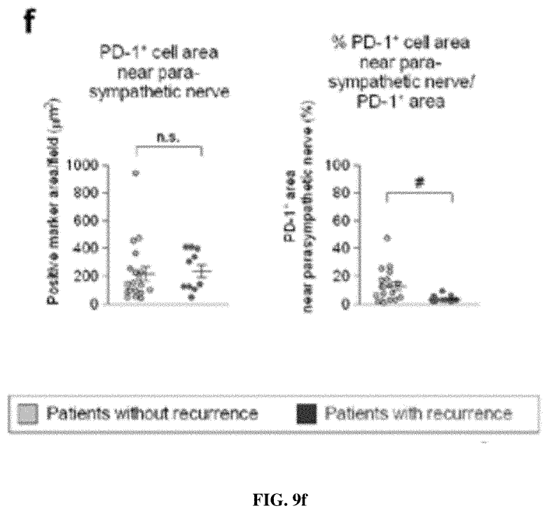

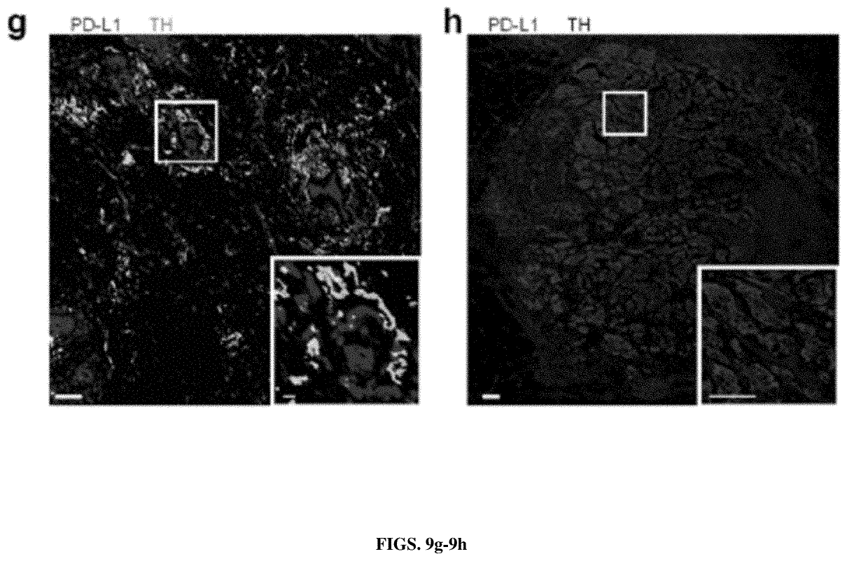

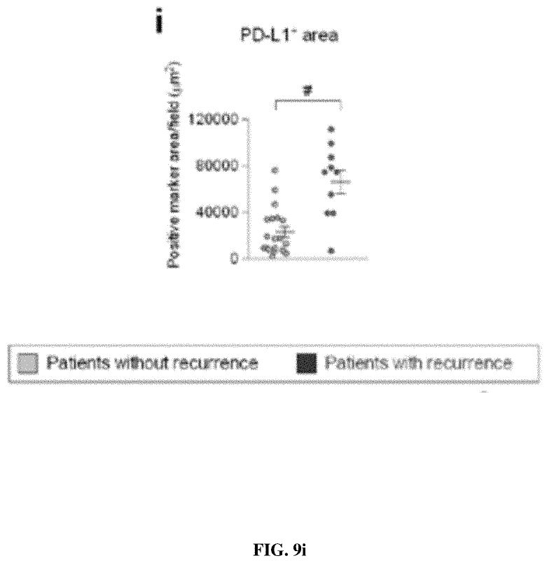

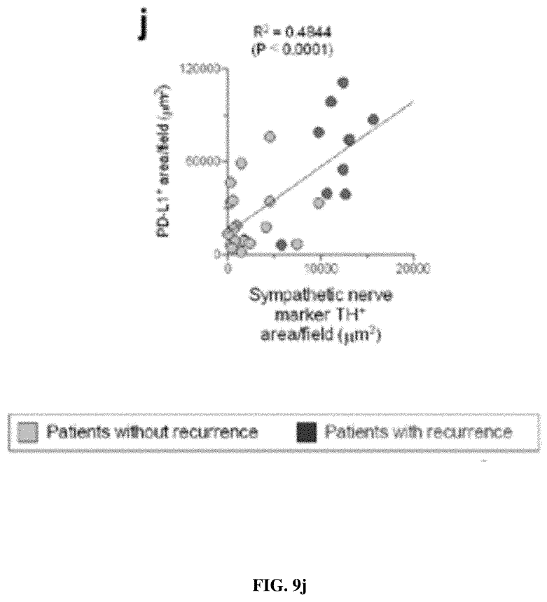

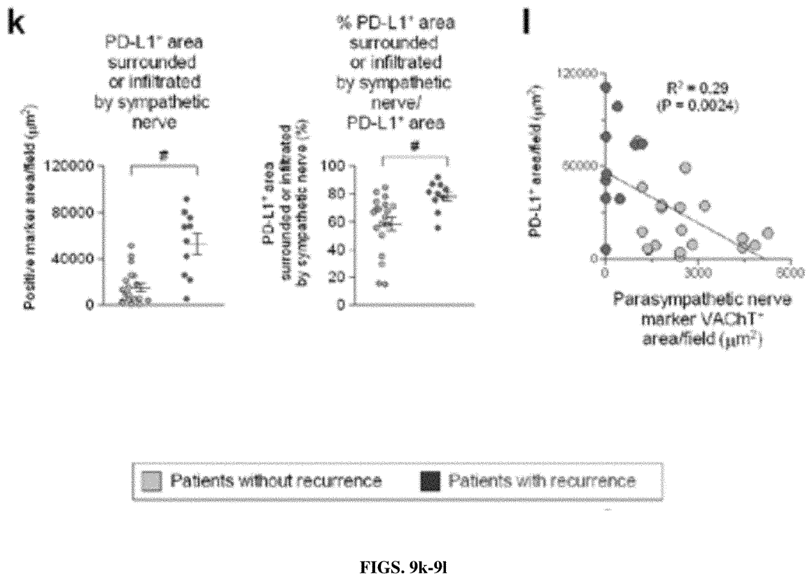

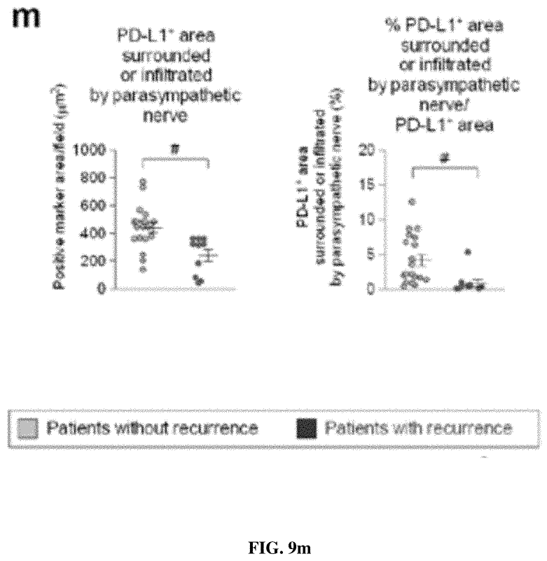

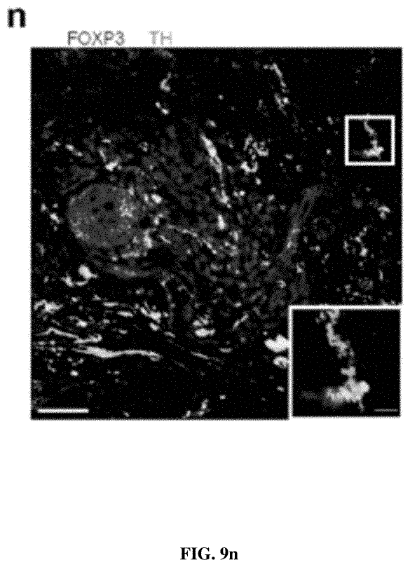

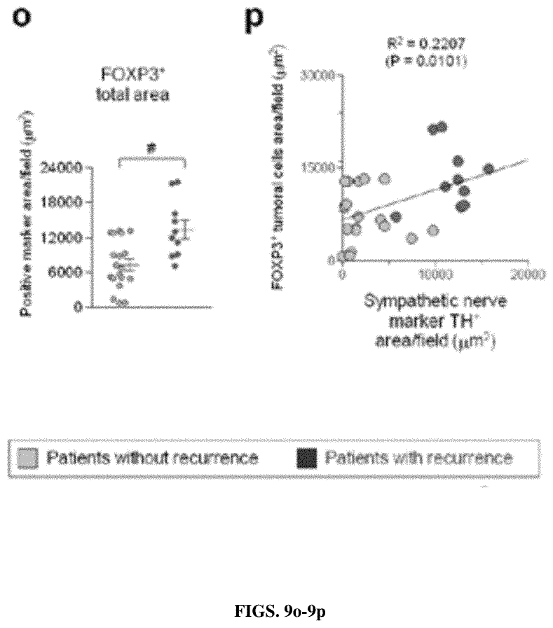

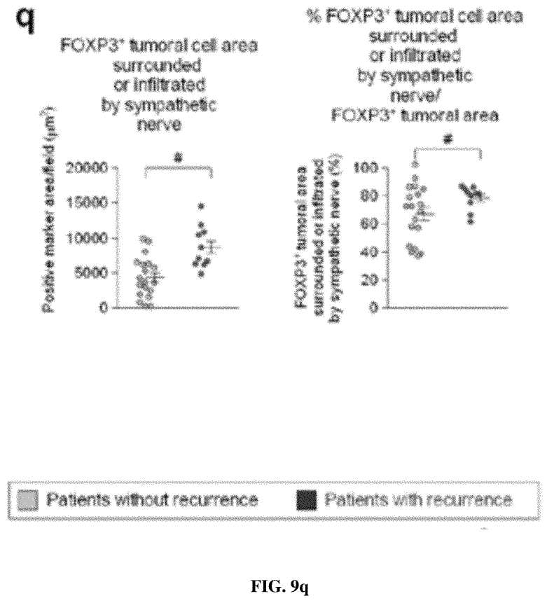

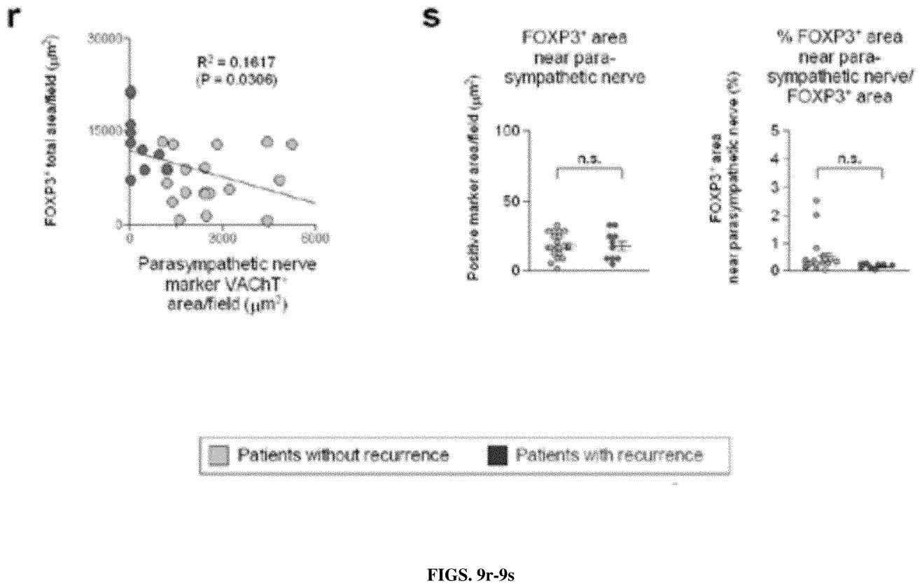

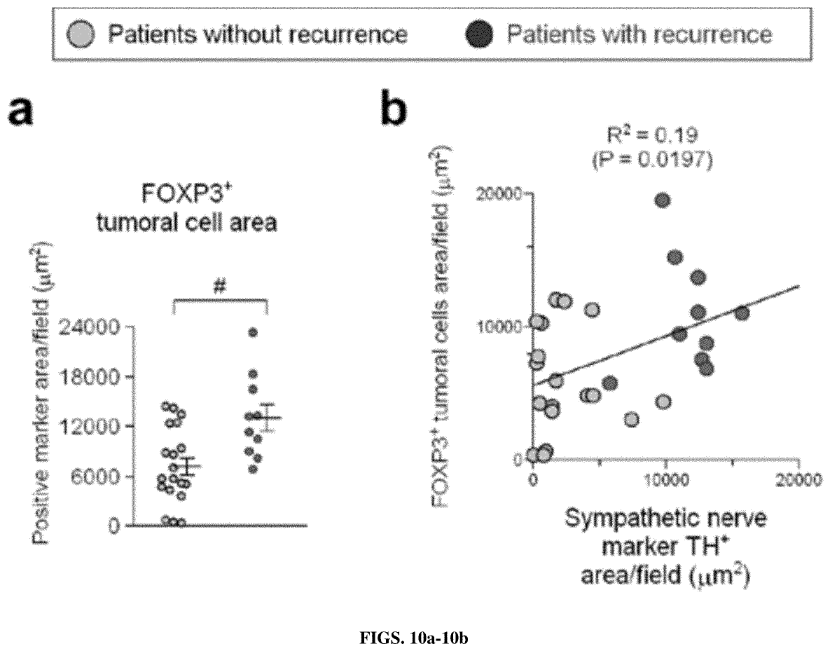

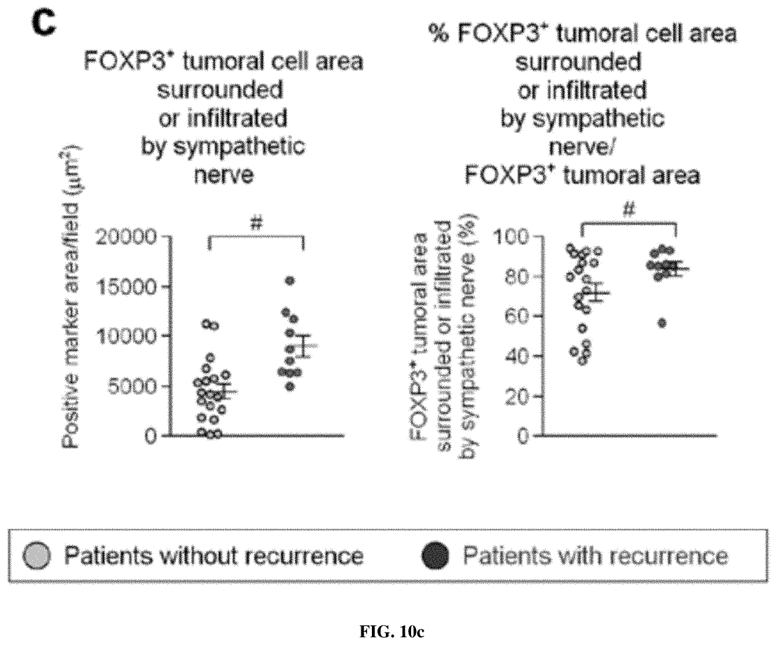

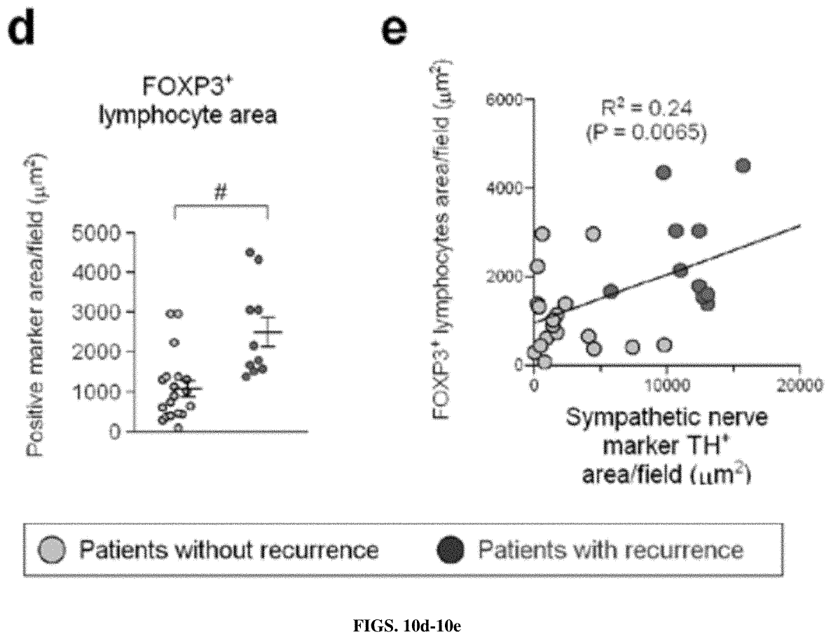

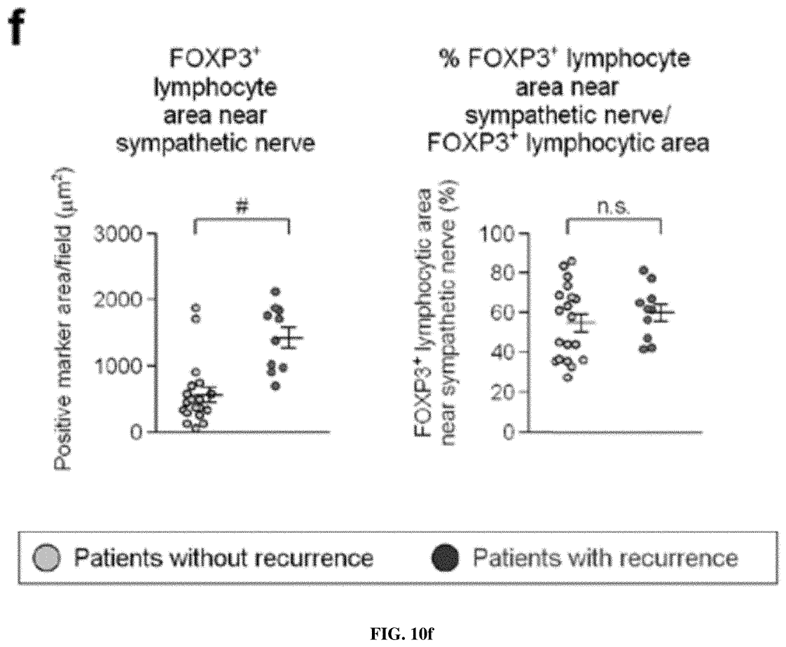

Next, because the present animal experiments revealed that deletion of sympathetic nerves and stimulation of parasympathetic nerves in the tumor microenvironment suppressed immune checkpoint molecules, tumor-infiltrating autonomic nerves were examined whether they are associated with the expression of immune checkpoint molecules in the human breast cancer specimens as shown in FIG. 9. First, as shown in FIG. 9a, immunofluorescence staining for PD-1 showed that the PD-1⁺ area (in putative lymphocytes) was greater in patients with recurrence than in those without as shown in FIG. 9b. The PD-1⁺ area positively correlated with the sympathetic nerve fiber density (P<0.0001, as shown in FIG. 9c) and negatively correlated with the parasympathetic nerve fiber density (P<0.005, as shown in FIG. 9e). Most (90%) of the PD-1⁺ area was close to or near tumor-infiltrating sympathetic nerves as shown in FIG. 9a in the boxed area, in patients with recurrence as shown in FIG. 9d, compared to those without (41%, P<0.001, as shown in FIG. 9d). In contrast to sympathetic nerves, PD-1⁺ cells were rarely (<15%) near parasympathetic nerves in patients, regardless of recurrence as shown in FIG. 9f, although slightly more were observed in patients without recurrence as shown in FIG. 9f. Next, immunofluorescence staining for PD-L1 as shown in FIG. 9g and FIG. 9h showed that the PD-L1⁺ area was greater in patients with recurrence than in those without as shown in FIG. 9i. The PD-L1⁺ area positively correlated with the sympathetic nerve fiber density (P<0.0001, as shown in FIG. 9j) and negatively correlated with the parasympathetic nerve fiber density (P<0.003, as shown in FIG. 9l). Most (78%) of PD-L1⁺ cells were surrounded as shown in FIG. 9g in the boxed area, or infiltrated as shown in FIG. 9h in the boxed area by sympathetic nerve fibers in patients with recurrence as shown in FIG. 9k, compared to those without (58%, P<0.005, as shown in FIG. 9k). In contrast to the sympathetic nerves, the PD-L1⁺ cells were rarely (<5%) surrounded or infiltrated by parasympathetic nerves, regardless of recurrence as shown in FIG. 9m, although slightly more were observed in patients without recurrence as shown in FIG. 9m. Lastly, immunofluorescence staining for FOXP3 as shown in FIG. 9n showed that the FOXP3⁺ area was greater in patients with recurrence than in those without as shown in FIG. 9o. The FOXP3⁺ area positively correlated with the sympathetic nerve fiber density (P=0.010, as shown in FIG. 9p) and negatively correlated with the parasympathetic nerve fiber density (P=0.031, as shown in FIG. 9r). Most (79%) of the FOXP3⁺ area was close to sympathetic nerve fibers in patients with recurrence as shown in FIG. 9q, compared to those without (67%, P<0.005, as shown in FIG. 9q). When the FOXP3⁺ area was putatively divided into tumoral cells and lymphocytes as shown in FIG. 10, these subpopulations were also higher in patients with recurrence as shown in FIG. 10a and FIG. 10d, positively correlated with sympathetic nerve fiber density as shown in FIG. 10b and FIG. 10e, and were surrounded by or infiltrated with these nerves as shown in FIG. 10c and FIG. 10f. In contrast to sympathetic nerves, the FOXP3⁺ area was not located near the parasympathetic nerves as shown in FIG. 9s. Together, these results shows that greater densities of sympathetic nerve fibers and lower densities of parasympathetic nerve fibers were associated with higher expression of immune checkpoint molecules (PD-1, PD-L1, and FOXP3), which were frequently in contact with or close to sympathetic nerve fibers in the human breast tumor microenvironment.

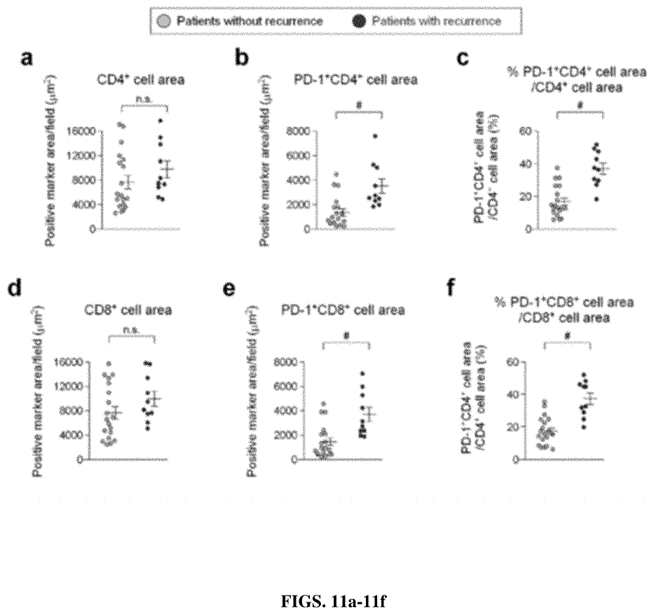

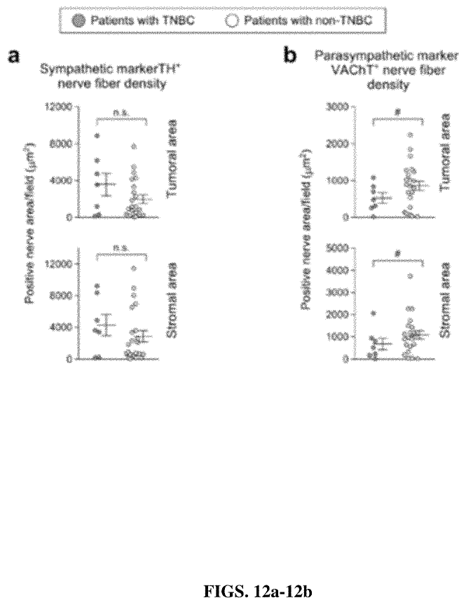

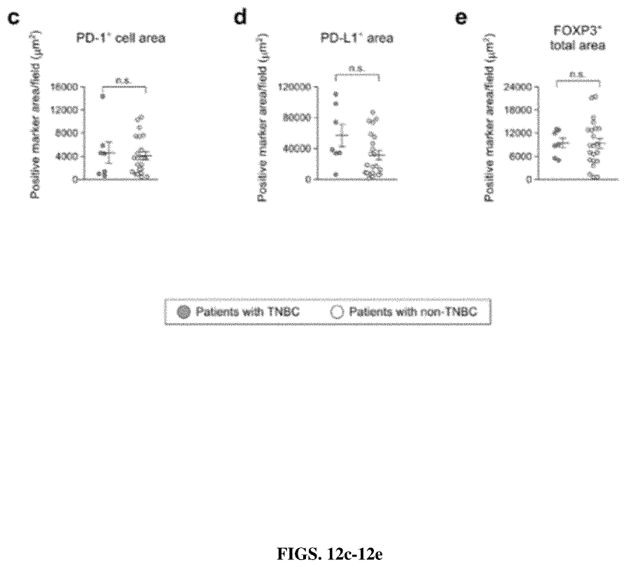

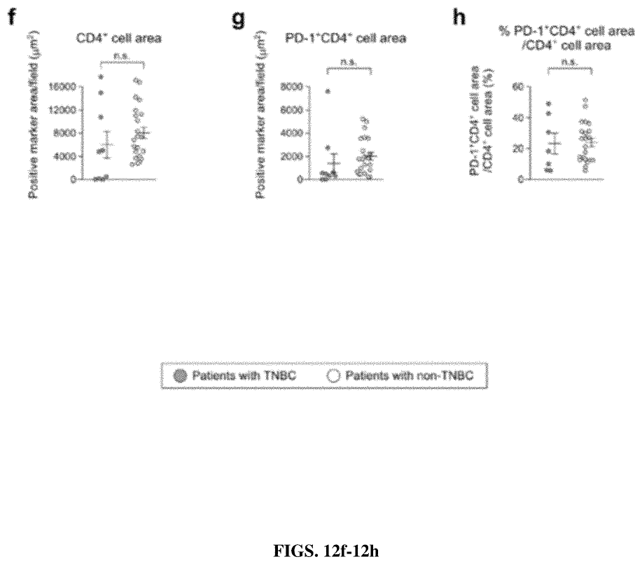

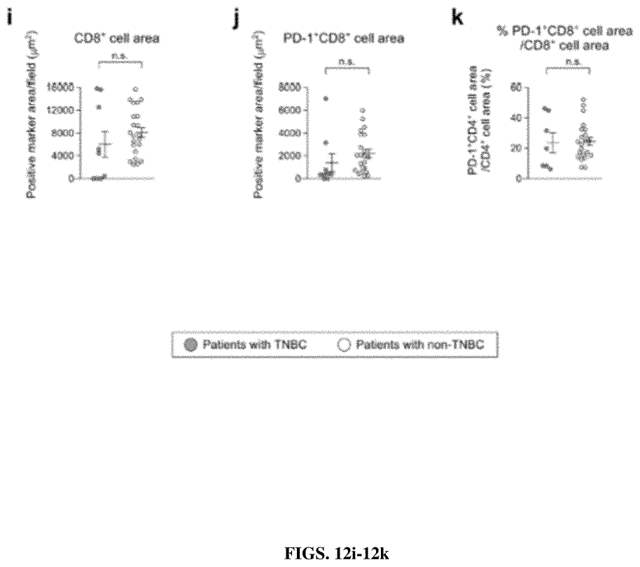

Next, immunofluorescence staining for PD-1 and CD4 as shown in FIG. 11a to FIG. 11c showed that PD-1⁺ CD4⁺ area as shown in FIG. 11b and percent expression of PD-1 on CD4⁺ cell (PD-1⁺ CD4⁺ cell area per CD4⁺ cell area) were greater in patients with recurrence than in those without as shown in FIG. 11c. Immunofluorescence staining for PD-1 and CD8 as shown in FIG. 11d to FIG. 11f showed that PD-1⁺ CD8⁺ area as shown in FIG. 11e and percent expression of PD-1 on CD8⁺ cell (PD-1⁺ CD8⁺ cell area per CD8⁺ cell area) were greater in patients with recurrence than in those without as shown in FIG. 11f. Lastly, there was no difference between patients with triple-negative breast cancer (TNBC) and non-TNBC in these parameters as shown in FIG. 12a to FIG. 12k; TH⁺ sympathetic as shown in FIG. 12a and VAChT⁺ parasympathetic nerve fiber densities as shown in FIG. 12b; PD-1⁺ cell area as shown in FIG. 12c; PD-L1⁺ cell area as shown in FIG. 12d; Foxp3⁺ cell area as shown in FIG. 12e; CD4⁺ cell area as shown in FIG. 12f, PD-1⁺ CD4⁺ cell area as shown in FIG. 12g and percent expression of PD-1 on CD4⁺ cell as shown in FIG. 12h; CD8⁺ cell area as shown in FIG. 12i, PD-1⁺ CD8⁺ cell area as shown in FIG. 12j and percent expression of PD-1 on CD8⁺ cell as shown in FIG. 12k.

As such, the present invention provides a superior efficacy in mediating tumor regression (via simultaneous inhibitory and conversely stimulatory action in a highly precise and localized manner as well as the ability to post-diagnostically modulate the ratio of this action in a tumor optimal manner). In addition, it provides reduced side effects in relation to existing therapies by means of its highly precise and highly local tumor targeting.

While this invention has been described in conjunction with the examples of embodiments outlined above, various alternatives, modifications, variations, improvements and/or substantial equivalents, whether know or that are or may be presently foreseen, may become apparent to those having at least ordinary skill in the art. Accordingly, the examples of embodiments of the invention, as set forth above, are intended to be illustrative, not limiting. Various changes may be made without departing from the spirit or scope of the invention. Therefore, the invention is intended to embrace all known or earlier developed alternatives, modifications, variations, improvements and/or substantial equivalents.

1. A method for the treatment of cancer comprising the step of administering a viral vector carrying a nerve deleting, nerve ablating or nerve inhibiting payload, the administration leading to the deletion, ablation or inhibition of tumor-specific sympathetic nerves.

2. A method for the treatment of cancer comprising the step of administering a viral vector carrying a nerve stimulating payload, resulting in the stimulation of tumor-specific parasympathetic nerves.

3. A method for the treatment of cancer comprising the step of administering a viral vector carrying a neurogenesis-promoting payload, resulting in an increased growth of tumor-specific parasympathetic nerves.

4. The method according to claim 1 further comprising the step of administering a viral vector carrying a nerve stimulating payload, resulting in the stimulation of tumor-specific parasympathetic nerves.

5. The method according to claim 1 further comprising the step of administering a viral vector carrying a neurogenesis-promoting payload, resulting in an increased growth of tumor-specific parasympathetic nerves.

6. The method according to claim 2 further comprising the step of administering a viral vector carrying a neurogenesis-promoting payload, resulting in an increased growth of tumor-specific parasympathetic nerves.

7. The method according to claim 4 further comprising the step of administering a viral vector carrying a neurogenesis-promoting payload, resulting in an increased growth of tumor-specific parasympathetic nerves.

8. The method according to claim 1, further comprising the step of administering a viral vector carrying a nerve deleting, nerve ablating or nerve inhibiting payload, the administration leading to the deletion, ablation or inhibition of tumor-specific afferent nerves.

9. The method according to claim 1, wherein the inhibition or stimulation of the nerve is achieved by chemogenetic methods.

10. The method according to claim 1 wherein the nerve deleting, nerve ablating or nerve inhibiting payload is a diphtheria toxin A subunit (DTA).

11. The method according to claim 2, wherein the nerve stimulating payload is AAV-ChAT-NachBac T220A-2A-GCaMP3.

12. The method according to claim 1, wherein the step of administering the viral vector is carried out by intratumoral injection.

13. The method according to claim 1, wherein the method is carried out in combination with other pharmacological agents which beneficially modulate an autonomic nervous system with respect to specific tumor types.

14. The method according to claim 1, wherein the method is carried out in combination with an administration of anti-seizure medications.

15. The method according to claim 14, wherein the method inhibits the autonomic nervous system and exerts additional anti-tumorigenic effects.

16. The method according to claim 1, wherein the method is carried out in combination with an administration of an anti-epileptic diet proven effective in patients resistant to anti-epileptic medications.

17. The method according to claim 1, wherein the method is carried out in combination with a use of medical instruments which beneficially modulate the autonomic nervous system with respect to specific tumor types.

18. The method according to claim 1, wherein the method is carried out in combination with an administration of conventional chemotherapeutic oncologic agents or acceptable pharmacological agents and radiological treatments.

19.-24. (canceled)
